(19) United States
(12) Patent Application Publication
(10) Pub. No.: US 2026/0103729 A1
(43) Pub. Date: Apr. 16, 2026

(54) rAAV PRODUCTION IN INSECT CELLS

(71) Applicant: Adverum Biotechnologies, Inc., Redwood City, CA (US)

(72) Inventor: Ali Aghajanirefah, Redwood City, CA (US)

(21) Appl. No.: 19/310,594

(22) Filed: Aug. 26, 2025

Related U.S. Application Data
(63) Continuation of application No. PCT/US2024/019049, filed on Mar. 8, 2024.
(60) Provisional application No. 63/489,656, filed on Mar. 10, 2023.
(60) Provisional application No. 63/465,669, filed on May 11, 2023.

(51) Int. Cl. C12N 15/86

(52) U.S. Cl. CPC C12N 15/86; C12N 2710/14022; C12N 2710/14043; C12N 2710/14052; C12N 2750/14122; C12N 2750/14144

(57) ABSTRACT

Materials for efficient production of recombinant baculovirus seed stocks containing a nucleic acid sequence that encodes a gene of interest and methods of producing recombinant baculovirus seed stocks containing a nucleic acid sequence that encodes a gene of interest are provided. Also provided are rapid methods of producing rAAV comprising a nucleic acid having a nucleic acid sequence that encodes a gene of interest; the method allows production of rAAV at a high titer.

### CROSS REFERENCE TO RELATED APPLICATIONS

This application claims priority to International Patent Application Number PCT/US2024/019049; which claims priority under 35 U.S.C. § 119(e) to U.S. Provisional Application No. 63/489,656 filed Mar. 10, 2023 and U.S. Provisional Application No. 63/465,669 filed May 11, 2023; each of which is incorporated by reference in its entirety.

### FIELD OF THE INVENTION

The present invention relates to the field of production of recombinant AAV, particularly high numbers of rAAV particles.

### BACKGROUND OF THE INVENTION

AAV is a 4.7 kb, linear, single-stranded DNA virus. rAAV is associated with excellent clinical safety, since wild-type AAV is non-pathogenic and has no etiological association with any known disease. Recent clinical and non-clinical success with recombinant adeno-associated virus (rAAV) vector as an in vivo gene delivery vehicle has increased interest in producing high purity and efficacious viral vectors for various natural and engineered serotypes.

The wild-type AAV genome encodes three open reading frames, rep, cap, and AAP, flanked with inverted terminal repeats (ITRs) which helps for replication, packaging, and rescue. AAV enters host cells via specific receptors on the cell surface.

The rep open reading frames codes for four rep proteins (Rep78, Rep68, Rep52, and Rep40) that are synthesized from mRNAs transcribed from the p5 and p19 promoters. Rep78 and Rep68 are required for adeno-associated virus (AAV) DNA replication. Rep52 and Rep40 contain helicase activity and are required for packaging AAV DNA into the capsids. VP1, VP2, and VP3 are synthesized from mRNA transcribed from the p40 promoter. To maintain a 1:1:10 ratio of VP1:VP2:VP3 for virus particle assembly, AAV uses an alternative splicing mechanism for VP1 and a less efficient start codon (ACG) for VP2 to lower their protein levels, yet keeps a high efficiency start codon for VP3. The N-terminal sequence present in VP1 contains a phospholipase A2 domain that is required for AAV infectivity. In addition, the VP2/VP3 mRNA codes for an assembly-activating protein (AAP) from a weak CTG start codon but in a different reading frame. AAP facilitates nuclear import of the major VP3 capsid protein and promotes assembly and maturation of the capsid, but AAP is not present in the mature capsid.

AAV vectors used for administration of therapeutic nucleic acids typically have approximately 96% of the parental genome deleted, such that only the terminal repeats (ITRs) which contain recognition signals for DNA replication and packaging remain.

This reduces immunologic or toxic side effects due to expression of viral genes. In addition, delivering specific AAV proteins to producing cells enables integration of the AAV vector comprising AAV ITRs into a specific region of the cellular genome if desired. See for example U.S. Pat. Nos. 6,342,390 and 6,821,511. Host cells comprising an integrated AAV genome show little or no change in cell growth or morphology (see, for example, U.S. Pat. No. 4,797,368).

Inverted terminal repeats (ITRs) flank the unique coding sequences for the non-structural replication (Rep) and s the structural capsid (Cap) proteins (also known as virion proteins (VPs). The ITRs are self-complementary and organized such that an energetically stable intramolecular duplex T-shaped hairpin can be formed. The hairpin structures function as an origin for viral DNA replication. The ITR is a site having a cis role in replication and serves as a recognition site for trans acting replication proteins.

One exception to the symmetry of the ITR sequence occurs at the “D” region of the ITR. Nicking of single-stranded DNA occurs at the junction between the A and D regions; it is the region where new DNA synthesis begins. The Rep genes encode the Rep proteins, Rep78, Rep68, Rep 52 and Rep40. The Cap genes encode the VP proteins, VP1, VP2 and VP3. These proteins form the capsid. An AAV replicating in a mammalian cell typically has two ITR sequences. AAV requires expression of helper genes for efficient replication.

Several AAV serotypes and over 100 AAV variants have been isolated from adenovirus stocks or from human or non-human primate tissues. Generally, the AAV serotypes have genomic sequences of significant homology at the nucleic acid sequence and amino acid sequence levels, such that different serotypes have an identical set of genetic functions, produce virions which are physically and functionally equivalent, and replicate and assemble by nearly identical mechanisms.

AAV Rep and ITR sequences are particularly conserved across many AAV serotypes. Moreover, the Rep sequences and ITRs of many AAV serotypes are known to efficiently cross-complement (i.e., functionally substitute) corresponding sequences from other serotypes during production of AAV particles in mammalian cells. Generally, the Cap proteins, which determine the cellular tropicity of the AAV particle and related Cap protein-encoding sequences, are significantly less conserved than Rep genes across different AAV serotypes. In view of the ability of Rep and ITR sequences to cross-complement corresponding sequences of other serotypes, the AAV vector can comprise a mixture of serotypes and thereby be a “chimeric” or “pseudotyped” AAV vector. A chimeric AAV vector comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotypes are further described in for example U.S. Pat. No. 6,723,551; Flotte (2006) Mol Ther 13(1): 1-2; Gao et al (2004) J. Virol 6381-6388; Gao et al (2002) Proc Natl Acad Sci USA 99:11854-11859; De et al (2006) Mol Ther 13:67-76 and Gao et al (2006) Mol Ther 13:77-87.

The most commonly used method for production of recombinant adeno-associated virus (rAAVs) in research laboratories is by transient triple transfection of 293 cells with AAV cis and trans plasmids and an adenovirus helper plasmid. However, most of the currently used HEK293 manufacturing platforms yield low titers and are not scalable. The difficulties associated with scaling-up rAAV production using mammalian cell production systems can be significant, if not entirely prohibitive. For example, a clinical study may require more than 10¹⁵ rAAV particles. To produce this number of rAAV particles in a mammalian cell line such as human 293 cells, transfection and culture of approximately 10¹¹ cells, the equivalent of 5,000 175 ml flasks of cells would be required. There is also a concern that a vector destined for clinical use produced in mammalian cell culture will be contaminated with undesirable, possibly pathogenic material from a mammalian cell.

U.S. Pat. No. 6,723,551 B2 to Kotin et al, and Urabe et al 2006 J. Virol. 80: 1874-1885 disclose methods of producing rAAV vectors in insect cells. AAV sequences employed for production of AAV in insect cells can be derived from the genome of any AAV serotype. Generally, the AAV serotypes have genomic sequences of significant sequence identity at the amino acid and nucleic acid levels, provide a virtually identical set of genetic functions, produce virions that are physically and functionally similar and replicate and assemble by practically identical mechanisms. Kotin and Urabe describe baculovirus vectors constructed with nucleic acids encoding Rep78/68 and Rep52/40 in palindromic head to tail arrangements under the control of an independent promoter.

These vectors include inverted terminal repeats (ITRs), gene of interest encoding sequences and AAV capsid genes. Although initial results suggested high-titer rAAV could be obtained with these methods, later work indicated the rAAV vectors were less infectious than counterparts produced in mammalian cells (Merten et al 2005 Gene Therapy 12:S51-S61 and Kohlbrenner et al 2005 Mol. Ther. 12:1217-1225).

The U.S. Pat. No. 6,723,551 suggests insect cells splice Rep mRNA differently than the process in mammalian cells. U.S. Pat. No. 6,723,551 avoids splicing out or removing an intron before translation.

In mammalian-cell produced AAV, the best yield of “full” virions (i.e., viral particles incorporating an AAV genome) that are fully functional and can target the nucleus is obtained when all three VP proteins are expressed at a stoichiometry approaching 1:1:10 (VP1:VP2:VP3). The splicing event required to produce a 1:1:10 stoichiometry of VP1:VP2:VP3 is not properly reproduced in insect cells when the original AAV Cap coding sequence is used. Modifications of the VP1 sequence have been used to modify the VP1 expression level. Attempts to use large homologous repeats of Rep78 and Rep52 indicated the homologous repeats were unstable. Attempts to separate the coding sequences for Rep78 and Rep52 required two baculoviruses and an additional baculovirus to supply VP1 protein. Cao et al 2000 J. Virol 74:11456-11463 described a method of producing high-titer, wild-type free recombinant AAV vectors using helper plasmids. However, Cao's methods used human cells and did not include a promoter that can direct transcription in insect cells.

U.S. Pat. No. 8,945,918 to Chen provides methods for expressing genes in insect cells and producing infectious rAAV vectors. The infectious rAAV vectors produced by Chen's methods exhibit loss of the gene of interest; by the second passage, only about 50% of the viral vectors contain the gene of interest. The viral particles continue to lose the gene of interest with each passage. By the third passage only about 25% of the viral particles contain the gene of interest. Less than 10% of the viral particles contain the gene of interest after the fourth passage. The methods described in U.S. Pat. No. 8,945,918 require multiple passages to obtain sufficient seed stock to produce large numbers of rAAV particles. High volumes of seedstock material are required for rAAV production with the Chen methods.

### SUMMARY OF THE INVENTION

Methods and systems of producing high-titer rAAV are provided herein. The rAAV produced by these methods and systems may be used in pharmaceutical compositions, gene therapy compositions, for research purposes and for other uses.

Systems for production of recombinant bacmids with reduced residual DNA in rAAV production are provided. The systems for production of recombinant bacmids with reduced residual DNA comprise (i) a donor plasmid comprising a first antibiotic resistance gene, a counter selective marker and a transfer cassette, wherein the transfer cassette comprises a left transposon arm, a gene of interest and a right transposon arm; (ii) a helper plasmid comprising a second antibiotic resistance gene; and (iii) a bacmid comprising a third antibiotic resistance gene and a transposon insertion site located within a reporter cassette.

In aspects of the system, each of the first, second and third antibiotic resistance genes confer resistance to a different antibiotic. In some aspects, the first antibiotic resistance gene, the second antibiotic resistance gene and the third antibiotic resistance gene are selected from the group comprising kanamycin resistance genes, ampicillin resistance genes, tetracycline resistance genes, penicillin resistance genes, streptomycin resistance genes, erythromycin resistance genes and penicillin/streptomycin resistance genes. In certain aspects, the first antibiotic resistance gene, the second antibiotic resistance gene and the third antibiotic resistance gene are selected from the group comprising ampicillin resistance gene, tetracycline resistance genes and kanamycin resistance genes. In an aspect, the bacmid comprises a kanamycin resistance gene, the helper plasmid comprises a tetracycline resistance gene and the donor plasmid comprises an ampicillin resistance gene and a counter selective marker that is rpsl.

In various aspects of the system, the counter selective marker is an antibiotic sensitive gene. In some aspects, the antibiotic sensitive gene is selected from the group comprising rpsl, URA3 and thymidine kinase. In certain aspects, the antibiotic sensitive gene is rpsl.

In some aspects, the donor plasmid does not comprise an active gentamycin resistance gene. In some aspects, the transfer cassette is optimized to reduce GC rich regions in the transfer cassette. In various aspects, the donor plasmid comprises neither an active gentamycin resistance gene nor a GC rich region in the transfer cassette.

The left transposon arm is Tn7R and the right transposon arm is Tn7L in some aspects of the system.

In aspects of the system, the reporter cassette comprises a lacZa gene. In some aspects, the transposon insertion site is selected from the group comprising a Tn7 insertion site, a modified Tn7 insertion site, and a mini-att-Tn7 site. In certain aspects, the transposon insertion site is a mini-att-Tn7 site.

In aspects of the system, the gene of interest encodes AAV RepCap proteins or a therapeutic gene product. In some aspects, the gene of interest that encodes AAV RepCap proteins comprises the 7m8 cassette.

In various aspects, the system is selected from the group comprising a modified Bac-to-Bac system, a modified baculovirus infectious cells (BIC) system and a modified Bac-plus system.

Methods of producing recombinant bacmids with reduced residual DN are provided. Methods of producing recombinant bacmids comprising reduced residual DNA comprise the steps of (a) introducing a system for production of recombinant bacmids comprising a gene of interest into bacterial cells, wherein the system comprises (i) a donor plasmid comprising a first antibiotic resistance gene, a counter selective marker and a transfer cassette, and wherein the transfer cassette comprises a left transposon arm, a gene of interest and a right transposon arm, (ii) a helper plasmid comprising a second antibiotic resistance gene, and (iii) a bacmid plasmid comprising a third antibiotic resistance gene and a transposon insertion site located with a reporter cassette; (b) growing the transformed bacteria in the presence of three antibiotics to which the first, second and third antibiotic resistance genes confer resistance; (c) selecting at least one bacterial colony comprising a recombinant bacmid; (d) incubating bacteria from at least one bacterial colony comprising a recombinant bacmid selected in step (c) with a compound that allows counter selection against the donor plasmid and (e) collecting recombinant bacmids with reduced residual DNA from a bacterial colony after counter selection.

In aspects of the methods, a bacterial colony comprising a recombinant bacmid is identified by an alteration in reporter activity. In various aspects, the reporter activity is selected from the group comprising fluorescence, antibiotic sensitivity and bacterial colony color. In some aspects of the methods, the transformed bacteria are grown in the presence of a beta-galactosidase substrate. In certain aspects, the beta-galactosidase substrate is selected from the group comprising x-gal and BluoGal. In various aspects, the presence of a recombinant bacmid is indicated by a white colony formed in the presence of the beta-galactosidase substrate.

In various aspects of the methods, each of the first, second and third antibiotic resistance genes confer resistance to a different antibiotic. In some aspects, the first antibiotic resistance gene, the second antibiotic resistance gene and the third antibiotic resistance gene are selected from the group comprising kanamycin resistance genes, ampicillin resistance genes, tetracycline resistance genes, penicillin resistance genes, streptomycin resistance genes, erythromycin resistance genes and penicillin/streptomycin resistance genes. In certain aspects, the first antibiotic resistance gene, the second antibiotic resistance gene and the third antibiotic resistance gene are selected from the group comprising ampicillin resistance gene, tetracycline resistance genes and kanamycin resistance genes. In an aspect, the bacmid comprises a kanamycin resistance gene, the helper plasmid comprises a tetracycline resistance gene and the donor plasmid comprises an ampicillin resistance gene and a counter selective marker that is rpsl. In some aspects, the compound that allows counter selection against the donor plasmid is an antibiotic. In certain aspects, the compound that allows counter selection against the donor plasmid is streptomycin.

Embodiments of the application provide efficient methods of producing a recombinant baculovirus seed stock comprising a gene of interest, wherein at least 50% of the recombinant baculovirus (rBV) in the recombinant baculovirus seed stock comprise a gene of interest. The efficient methods comprise the steps of (a) introducing a system for production of recombinant bacmids comprising a gene of interest into bacterial cells, wherein the system comprises (i) a donor plasmid comprising a first antibiotic resistance gene, a counter selective marker and a transfer cassette, and wherein the transfer cassette comprises a left transposon arm, a gene of interest and a right transposon arm; a helper plasmid comprising a second antibiotic resistance gene; and a bacmid comprising a third antibiotic resistance and a transposon insertion site located within a reporter cassette. The methods comprise the steps of (b) growing the transformed bacteria in the presence of three antibiotics to which the first, second and third antibiotic resistance genes confer resistance; (c) selecting at least on bacterial colony comprising a recombinant bacmid; (d) incubating a bacteria from at least one bacterial colony comprising a recombinant bacmid selected in step (c) with a compound that allows counter selection against the donor plasmid; (e) collecting recombinant bacmids comprising a gene of interest from a bacterial colony after counter selection; (f) transforming insect cells with the recombinant bacmids comprising a gene of interest collected in step (e); (g) incubating the transformed insect cells and harvesting rBV comprising the gene of interest from the insect cells; (h) performing a first passage, wherein a passage comprises the steps of infecting insect cells with rBV comprising the gene of interest and incubating the infected insect cells; (i) harvesting rBV comprising the gene of interest from at least two plaques of insect cells and identifying a plaque comprising rBV comprising a gene of interest and wherein the rBV exhibit a low rate of loss of the gene of interest; and performing a second passage (p1 rBV), wherein the second passage comprises infecting insect cells with rBV from the plaque identified in step (i), incubating the infected insect cells and harvesting recombinant baculovirus seed stock comprising rBV comprising the gene of interest from the insect cells.

In some aspects of the method, after the first passage, at least 80% of the rBV comprise the gene of interest. In various aspects of the method, after the second passage at least 70% of the rBV comprise the gene of interest. In certain aspects of the methods, the method produces a recombinant baculovirus seed stock after two passages through insect cells, wherein the baculovirus seed stock is at least 2×10¹⁰ vector genomes/ml (vg/ml).

In various aspects of the methods, a bacterial colony comprising a recombinant bacmid is identified by an alteration in reporter activity. In some aspects, the reporter activity is selected from the group comprising fluorescence, antibiotic sensitivity and bacterial colony color. In some aspects of the methods, the transformed bacteria are grown in the presence of a beta-galactosidase substrate. In certain aspects, the beta-galactosidase substrate is selected from the group comprising x-gal and BluoGal. In various aspects, the presence of a recombinant bacmid is indicated by a white colony formed in the presence of the beta-galactosidase substrate.

In various aspects of the methods, each of the first, second and third antibiotic resistance genes confer resistance to a different antibiotic. In some aspects, the first antibiotic resistance gene, the second antibiotic resistance gene and the third antibiotic resistance gene are selected from the group comprising kanamycin resistance genes, ampicillin resistance genes, tetracycline resistance genes, penicillin resistance genes, streptomycin resistance genes, erythromycin resistance genes and penicillin/streptomycin resistance genes. In certain aspects, the first antibiotic resistance gene, the second antibiotic resistance gene and the third antibiotic resistance gene are selected from the group comprising ampicillin resistance gene, tetracycline resistance genes and kanamycin resistance genes. In an aspect, the bacmid comprises a kanamycin resistance gene, the helper plasmid comprises a tetracycline resistance gene and the donor plasmid comprises an ampicillin resistance gene and a counter selective marker that is rpsl. In some aspects, the compound that allows counter selection against the donor plasmid is an antibiotic. In certain aspects, the compound that allows counter selection against the donor plasmid is streptomycin.

In some aspects of the methods, the step of incubating the transformed insect cells occurs at about 28° C. and without CO₂ for approximately four days. In various aspects of the methods, the step of incubating the transformed insect cells occurs in a shaker incubator. In certain aspects, the insect cells are grown in 30 ml of media.

In certain aspects of the methods, the insect cells are selected from the group comprising Sf-RVN cells, Sf9 cells and Hi5 cells. In an aspect, the insect cells are selected from the group comprising Sf-RVN cells and Sf9 cells.

In various aspects, the insect cells are transformed with between about 1 μg and 20 μg bacmids. In certain aspects, the insect cells are transformed with about 6-9 μg bacmids.

In some aspects the gene of interest encodes Rep/Cap. In aspects, the gene of interest that encodes Rep/Cap comprises the 7m8 cassette. In certain aspects, the gene of interest comprises the 7m8 cassette.

In particular aspects, the step of harvesting rBV comprising the gene of interest comprises harvesting rBV from at least ten plaques of insect cells and identifying a plaque comprising rBV having a stable gene of interest.

In some aspects, the methods further comprise performing a third passage, wherein the third passage (p2 rBV) comprises infecting insect cells with rBV from p1 rBV identified in step (j) incubating the infected insect cells and harvesting a recombinant baculovirus seed stock comprising rBVs comprising the gene of interest or Rep/Cap from the insect cells.

Methods of producing a recombinant baculovirus seed stock comprising a stable gene of interest, wherein at least 50% of the recombinant baculovirus (rBV) in the recombinant baculovirus seed stock comprise a stable gene of interest are provided. The methods comprise the steps of (a) introducing a system for production of recombinant bacmids comprising a gene of interest into bacterial cells, wherein the system comprises (i) a donor plasmid comprising a first antibiotic resistance gene, a counter selective marker, and a transfer cassette, and wherein the transfer cassette comprises a left transposon arm, a gene of interest and a right transposon arm; (ii) a helper plasmid comprising a second antibiotic resistance gene; and (iii) a bacmid comprising a third antibiotic resistance gene and a transposon insertion site located within a reporter cassette. The methods comprise the steps of (b) growing the transformed bacteria in the presence of three antibiotics to which the first, second and third antibiotic resistance genes confer resistance; (c) selecting at least one bacterial colony comprising a recombinant bacmid; (d) incubating bacteria from at least one bacterial colony comprising a recombinant bacmid selected in step (c) with a compound that allows counter selection against the donor plasmid; (e) collecting recombinant bacmids comprising a gene of interest from a bacterial colony after counter selection; (f) transforming insect cells with recombinant bacmids comprising a gene of interest collected in step (e); (g) incubating the transformed insect cells and harvesting rBV comprising the gene of interest from the insect cells; (h) performing a first passage, wherein a passage comprises the steps of infecting insect cells with rBV comprising the gene of interest and incubating the infected insect cells; (i) harvesting rBV comprising the gene of interest from one or more insect cell plaques and identifying a plaque comprising rBV's comprising a stable gene of interest; and (j) performing a second passage, wherein the second passage comprises infecting insect cells with rBV comprising a stable gene of interest, incubating the infected insect cells and harvesting a recombinant baculovirus seed stock comprising rBV comprising the stable gene of interest from the insect cells.

In an embodiment, a recombinant baculovirus seed stock comprising a stable gene of interest is provided.

In an embodiment, a recombinant baculovirus seed stock comprising a stable gene of interest produced by a method of the application is provided. In aspects of the recombinant baculovirus seed stock, the stable gene of interest comprises the 7m8 cassette. In aspects of the recombinant baculovirus seed stock, the stable gene of interest encodes Rep/Cap.

Compositions comprising a recombinant baculovirus seed stock comprising a stable gene of interest are provided.

Methods of producing rAAV comprising a gene of interest are provided. Methods of producing rAAV comprising at gene of interest comprise the steps of (a) producing a first recombinant baculovirus seed stock by an above method, wherein the first recombinant baculovirus seed stock comprises rBV comprising a first gene of interest; (b) producing a second recombinant baculovirus seed stock by an above method, wherein the second baculovirus seed stock comprises rBV encoding Rep/Cap proteins; (c) co-infecting insect cells with the first recombinant baculovirus seed stock and with the second recombinant baculovirus seed stock; (d) adding feed to the insect cell media after co-infection; (e) incubating the infected insect cells and (f) harvesting rAAV comprising the gene of interest from the insect cells. In aspects of the method, the step of co-infecting insect cells comprises coinfecting insect cells with a first recombinant baculovirus seed stock with a low multiplicity of infection (MOI) and with a second recombinant baculovirus seed stock with a low multiplicity of infection. In various aspects, the low MOI is below 0.01. In various aspects, the low MOI is below 0.009. In certain aspects, the low MOI is in the range of about 0.0005 to about 0.009. In certain aspects, the low MOI is in the range of about 0.001. In various aspects, the MOI of the first recombinant baculovirus seed stock differs from the MOI of the second recombinant baculovirus seed stock. In other aspects, the MOI of the first recombinant baculovirus seed stock and the MOI of the second recombinant baculovirus seed stock is the same.

In aspects of the method, the harvested rAAV has a titer of at least 111 vg/ml. In some aspects, the harvested rAAV has a titer of at least 511 vg/ml. In certain aspects, the harvested rAAV has a titer of at least 112 vg/ml.

In various aspects, the feed is added between about 1 hour and 8 hours after co-infection. In some aspects, the feed is added about 4 hours after co-infection.

In some aspects, the insect media is ESF-AF.

In various aspects, the insect cells are selected from the group comprising Sf-RVN cells, Sf9 cells and Hi5 cells. In certain aspects, the insect cells are Sf-RVN cells.

In some aspects of the methods, rAAV comprising a gene of interest are harvested at a high titer less than 25 days after transforming insect cells with recombinant bacmids. In some aspects, rAAV comprising a gene of interest are harvested at a high titer approximately 23 days after transforming insect cells with recombinant bacmids. In certain aspects, rAAV comprising a gene of interest are harvested at a high titer five days after co-infection. In other aspects, p2 rBV are harvested at high titer 18 days after transforming insect cells with recombinant bacmids.

In various aspects of the methods, the volume of either the first recombinant baculovirus seed stock, the second recombinant baculovirus seed stock or the volumes of each of the first recombinant baculovirus seed stock and the second recombinant baculovirus seed stock used to co-infect the insect cells is less than 1 liter. In other aspects, the volume of either the first recombinant baculovirus seed stock, the second recombinant baculovirus seed stock or the volumes of each of the first recombinant baculovirus seed stock and the second recombinant baculovirus seed stock used to co-infect the insect cells is less than 500 ml. In some aspects, the volume of either the first recombinant baculovirus seed stock, the second recombinant baculovirus seed stock or the volumes of each of the first recombinant baculovirus seed stock and the second recombinant baculovirus seed stock used to co-infect the insect cells is less than 100 ml. the volume of either the first recombinant baculovirus seed stock, the second recombinant baculovirus seed stock or the volumes of each of the first recombinant baculovirus seed stock and the second recombinant baculovirus seed stock used to co-infect the insect cells is less than 30 ml.

Methods of rapidly producing rAAV comprising a gene of interest comprising the steps of (a) producing a first recombinant baculovirus seed stock by a method described above herein, wherein the first recombinant baculovirus seed stock comprises rBV comprising a first gene of interest; (b) producing a second recombinant baculovirus seed stock by a method described above herein, wherein the second recombinant baculovirus seed stock comprises rBV encoding Rep/Cap proteins; transducing insect cells with the first recombinant baculovirus seed stock at a low multiplicity of infection and with the second recombinant baculovirus seed stock at a low MOI; (d) adding feed to the insect cell media after co-infection; (e) incubating the infected insect cells for five days and (f) harvesting rAAV comprising the first gene of interest from the insect cells less than 27 days after transforming insect cells with recombinant bacmids.

In various aspects of the methods, the first gene of interest encodes a therapeutic gene product for use in treating ocular disease or disorders. In certain aspects, the ocular disease or disorder is selected from the group comprising glaucoma, retinitis pigmentosa, macular degeneration, retinoschisis, Leber's Congenital Amaurosis, diabetic retinopathy, achromotopsia and color blindness. In some aspects, the macular degeneration is selected from the group comprising dry macular degeneration, wet macular degeneration and age-related macular degeneration. In some aspects of the methods, the therapeutic gene product is selected from the group comprising an anti-angiogenic polypeptide, a vascular endothelial growth factor (VEGF) binding protein, an opsin protein, an anti-C3 antibody, an anti-C5 antibody, complement factor I (CFI) and an anti-dry AMD gene product. In certain aspects of the methods, the therapeutic gene product is selected from the group comprising aflibercept, sFLT1, CFI, ranibizumab, and bevacizumab.

In an embodiment, methods of rapidly producing rAAV comprising a gene of interest are provided. The methods of rapidly producing rAAV comprising a gene of interest comprise the steps of (a) producing a first recombinant baculovirus seed stock wherein the first baculovirus seed stock comprises rBV comprising a first gene of interest; (b) producing a second recombinant baculovirus seed stock, wherein the second recombinant baculovirus seed stock comprises rBV encoding Rep/Cap proteins; (c) transducing insect cells with the first recombinant baculovirus seed stock at a low MOI and with the second recombinant baculovirus seed stock at a low MOI; (d) adding feed to the insect cell media about four hours after co-infection; (e) incubating the infected insect cells for five days; and (f) harvesting rAAV comprising the first gene of interest from the insect cells five days post-co-infection.

In an embodiment, methods of producing rAAV comprising a gene of interest are provided. The methods comprise the steps of (a) producing a first recombinant baculovirus seed stock, wherein the first recombinant baculovirus seed stock comprises rBV comprising a first gene of interest; (b) providing a second recombinant baculovirus seed stock comprising rBV comprising a stable gene of interest encoding Rep/Cap proteins; (c) co-infecting insect cells with the first recombinant baculovirus seed stock at a low MOI and with the second recombinant baculovirus seed stock at a low MOI; (d) adding feed to the insect cell media after co-infection; incubating the infected insect cells; and harvesting rAAV comprising the gene of interest from the insect cells. In various aspects, the second recombinant baculovirus seed stock comprises rBV comprising the 7m8 cassette.

In an embodiment, a composition comprising recombinant bacmids with reduced residual DNA produced by a method described above herein are provided.

Methods of producing rAAV comprising a gene of interest comprising the steps of (a) providing a first recombinant baculovirus seed stock comprising rBV comprising a stable first gene of interest; (b) providing a second recombinant baculovirus seed stock comprising rBV comprising a stable gene of interest encoding Rep/Cap proteins; (c) co-infecting insect cells with the first recombinant baculovirus seed stock at a low MOI and with the second recombinant baculovirus seed stock at a low MOI; (d) adding feed to the insect cell media after co-infection; (e) incubating the infected insect cells; and (f) harvesting rAAV comprising the gene of interest from the insect cells.

Methods of rapidly producing rAAV comprising a gene of interest are provided. The methods of rapidly producing rAAV comprising a gene of interest comprise the steps of (a) producing a first recombinant baculovirus seed stock comprising rBV comprising a first gene of interest; (b) providing a second recombinant baculovirus seed stock comprising rBV comprising a stable gene of interest encoding Rep/Cap proteins; (c) co-infecting insect cells with the first recombinant baculovirus seed stock at a low MOI and with the second recombinant baculovirus seed stock at a low MOI; (d) adding feed to the insect cell media after co-infection; (e) incubating the infected insect cells; and (f) harvesting rAAV comprising the gene of interest from the insect cells approximately five days post co-infection. The steps of producing a first or second recombinant baculovirus seed stock may utilize a method described above herein.

In an embodiment, compositions comprising recombinant bacmids with reduced residual DNA produced by a method of the application are provided. In an aspect, a composition comprising recombinant bacmids with reduced residual DNA is for use in production of a pharmaceutical composition.

In an embodiment, an insect cell capable of producing rAAV comprising a stable gene of interest is provided.

In various systems, the recombinant bacmids with reduced residual DNA are for use in rAAV production. In aspects of the systems, the helper plasmid is an AAV helper plasmid.

Modified Bac-to-Bac systems for production of recombinant bacmids with reduced residual DNA in rAAV production are provided herein. The modified Bac-to-Bac systems comprise a donor plasmid comprising a first antibiotic resistance gene, a counter selective marker and a transfer cassette. The donor plasmid does not comprise an active gentamycin resistance gene and a GC rich region in the transfer cassette. The transfer cassette comprises a left transposon arm, a gene of interest and a right transposon arm. The modified Bac-to-Bac systems comprise an AAV helper plasmid comprising a second antibiotic resistance gene. The modified Bac-to-Bac systems comprise a bacmid comprising a third antibiotic resistance gene and a transposon insertion site located within a reporter cassette. In some aspects of the systems, the first antibiotic resistance gene, the second antibiotic resistance and the third antibiotic resistance gene are selected from the group comprising an ampicillin resistance gene, a tetracycline resistance gene and a kanamycin resistance gene. In aspects of the systems, the bacmid comprises a kanamycin resistance gene, the helper plasmid comprises a tetracycline resistance gene and the donor plasmid comprises an ampicillin resistance gen and a counter selective marker that is rpsl. In some aspects, the counter selective marker is rspl. Aspects of the systems include wherein the left transposon arm is Tn7R and the right transposon arm is Tn7L. In various aspects of the systems, the reporter cassette comprises a lacZa gene. In certain aspects of the systems, the transposon insertion site is a mini-att-Tn7 site. In various aspects of the system, the gene of interest encodes AAV RepCap proteins or a therapeutic gene product.

In an embodiment, the application provides efficient methods of producing a recombinant baculovirus seed stock comprising a gene of interest, wherein at least 50% of the recombinant baculovirus (rBV) in the recombinant baculovirus seed stock comprise a gene of interest. In aspects of the methods, after two passages at least 70% of the rBV comprise the gene of interest. In some aspects, after one passage at least 80% of the rBV comprise the gene of interest. In certain aspects, the method produces a recombinant baculovirus seed stock after two passages through insect cells wherein the recombinant baculovirus seed stock is at least 2×10¹⁰ vector genomes/ml (vg/ml).

Some aspects of the efficient methods of producing a recombinant baculovirus seed stock comprising a gene of interest comprise the step of introducing a modified Bac-to-Bac system for production of recombinant rBV comprising a gene of interest into bacterial cells, wherein the modified Bac-to-Bac system comprises (i) a donor plasmid comprising a first antibiotic resistance gene, a counter selective marker and a transfer cassette, wherein the donor plasmid comprises neither an active gentamycin resistance gene nor a GC rich region in the transfer cassette and wherein the transfer cassette comprises a left transposon arm, a gene of interest and a right transposon arm; (ii) a helper plasmid comprising a second antibiotic resistance gene and (iii) a bacmid plasmid comprising a third antibiotic resistance gene. The efficient methods further comprise the steps of (b) growing the transformed bacteria in the presence of three antibiotics to which the first, second and third antibiotic resistance genes confer resistance, IPTG and BluoGal; (c) selecting at least one bacterial colony comprising a recombinant bacmid (white colony); (d) incubating bacteria from at least one bacterial colony comprising a recombinant bacteria selected in step (c) with a compound that allows counter selective against the donor plasmid; (e) collecting bacmids from a bacterial cell comprising a recombinant bacmid; (f) transforming insect cells with the recombinant bacmids collected in step (e); (g) incubating the transformed insect cells in media at 28° C. for four days in a shaker incubator and collecting PO recombinant baculovirus (rBv, BEVs); (h) performing a first passage wherein a passage comprises the steps of infecting insect cells with PO recombinant baculovirus (rBv) comprising the gene of interest or Rep/Cap and incubating the infected insect cells; (i) harvesting RBV comprising the gene of interest from at least ten plaques of insect cells and selecting the most stable plaque; and (j) performing a second passage, wherein the second passage comprises infecting insect cells with rBV from the plaque purification step (step i), incubating the infected insect cells and harvesting a recombinant baculovirus seed stock comprising p1 rBV comprising the gene of interest from the insect cells. In some aspects, the presence of a recombinant bacmid in a bacterial cell is indicated by a white colony. In certain aspects of the methods, the donor plasmid comprises an ampicillin resistance gene and rpsl, the helper plasmid comprises a tetracycline resistance gene and the bacmid plasmid comprises a kanamycin resistance gene. In various aspects of the methods, the compound that allows counter selective against the donor plasmid is streptomycin. In some aspects, the insect cells are selected from the group comprising Sf-RVN cells and Sf9 cells. In certain aspects of the methods, the insect cells are transformed with about 6-9 μg bacmids. In aspects of the methods, the transformed insect cells are grown in 30 ml of media. In some aspects, the methods further comprise performing a third passage, wherein the third passage comprises infecting insect cells with BEVs from the P1 rBV step (step j), incubating the infected insect cells and harvesting a baculovirus seed stock comprising BEVS comprising the gene of interest from the insect cells (P2 rBV). In some case for example in the prevalent disease, P2 rBV may be re-plaqued to regenerate starting viral bank at P1 rBV. In some aspects of the third passage, the MOI is 0.1.

In an embodiment, the application provides methods of producing rAAV comprising a gene of interest comprising the steps of (a) producing a first recombinant baculovirus seed stock by a method described herein, wherein the first recombinant baculovirus seed stock comprises rBV comprising a first gene of interest, (b) producing a second recombinant baculovirus seed stock by the method of claim 13, wherein the second recombinant baculovirus seed stock comprises rBV encoding Rep/Cap proteins; (c) co-infecting insect cells with the first recombinant baculovirus seed stock at a low multiplicity of infection (MOI) and with the second recombinant baculovirus seed stock at a low MOI; (d) adding feed to the insect cell media after co-infection with the first recombinant baculovirus seed stock and the second recombinant baculovirus seed stock; (e) incubating the infected insect cells and harvesting rAAV comprising the gene of interest from the insect cells. In aspects of the methods, the low MOI is in the range of between about 0.01 and 0.001. In aspects of the methods, the low MOI is in the range of about 0.001. In various aspects of the methods, the harvested rAAV has a titer of at least 112 vg/ml. In certain aspects, the feed is added about four hours after co-infection. In some aspects of the methods, the insect media is ESF-AF. In certain aspects, the insect cells are selected from the group comprising Sf-RVN cells and Sf9 cells. In aspects of the methods, rAAV comprising a gene of interest are harvested at a high titer five days after co-infection. In aspects of the methods, p2 rBV are harvested at a high titer 18 days after transforming insect cells with bacmids collected from a bacterial colony comprising a recombinant bacmid. In various aspects of the methods, the volume of either the first recombinant baculovirus seed stock, the second recombinant baculovirus seed stock or both the first recombinant baculovirus seed stock and second recombinant baculovirus seed stock used to coinfect the cells is less than 30 ml.

In an embodiment, methods of rapidly producing rAAV comprising a gene of interest are providing. The methods comprise the steps of (a) producing a first recombinant baculovirus seed stock by a method described herein, wherein the first recombinant baculovirus seed stock comprises rBV comprising the first gene of interest, (b) producing a second recombinant baculovirus seed stock by a method described herein, wherein the second recombinant baculovirus seed stock comprises rBV encoding Rep/Cap proteins, (c) transducing insect cells with the first recombinant baculovirus seed stock at a low multiplicity of infection and with the second recombinant baculovirus seed stock at a low multiplicity of infection, (d) adding feed to the insect cell media about four hours after co-infection with the first recombinant baculovirus seed stock and the second recombinant baculovirus seed stock, (e) incubating the infected insect cells for five days and harvesting rAAV comprising a gene of interest from insect cells five days post-co-infection.

### INCORPORATION BY REFERENCE

The following passages describe different aspects of the invention in greater detail. Each aspect, embodiment, or feature of the invention may be combined with any other aspect, embodiment, or feature the invention unless clearly indicated to the contrary. All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF DRAWINGS

FIG. 1 provides an overview of the modifications to the donor plasmid and the process for obtaining recombinant bacmids without the donor plasmid (pFastBac) in a modified bac-to-bac process. Media contains the indicated additives. The modified bac-to-bac process is one example of a system for producing recombinant bacmids with reduced residual DNA.

FIG. 2 provides images of E. coli colonies grown under various conditions. The modified Bac-to-Bac system allows selection of colonies lacking the donor plasmid (2) and with reduced residual DNA in the bacmids collected from bacteria after incubation of the bacteria with a compound that allows counter selective against the donor plasmid.

The donor plasmid (pFastBac) is significantly not visible in lane 2 of the gel, indicating a significant reduction of the donor plasmid.

FIG. 3 provides a schematic summarizing the prior process of obtaining rAAV from bacmids in comparison with the current (new) process. The new methods eliminate required steps. The recombinant bacmids produced by the systems of the current application have reduced residual DNA.

FIG. 4 provides images of plates of infected cells obtained during the plaque purification step for two different rBV. The two different rBV comprise different expression cassettes (CFI 1.0 and 7m8) comprising either CFI 1.0 as the gene of interest or comprising 7m8 Rep/Cap as the gene of interest. The plaque purification process depicted herein is similar to that described in Example 4. The images depicted show plates with a control and 10¹, 10⁻², 10⁻⁴, 10⁻⁶, and 10⁻⁸ dilutions.

FIG. 5 provides a schematic overview of the process of producing rAAV using a modified Bac-to-Bac system contrasted with the standard production process. Note the shorter time-line from bacmid to harvesting of rAAV from Sf-RVN cells using the modified system versus the traditional methods. The Sf-RVN cells are rhabdovirus free.

FIG. 6 provides charts summarizing the rBaculovirus (BEV) stability during different passages and its effect on rAAV titer. The rBV comprising 7m8 were obtained from two different plaques (plaque 1 and plaque 2). The ratio of the gene of interest (the 7m8 Rep/Cap cassette) to GP64 is indicated for rBV from each plaque at P0, Passage 1 (P1) and Passage 2 (P2). The rBV from plaque 2 have a gene of interest to backbone ratio equal to or greater than 0.5 after Passage 2. The rBV from plaque 2 have a stable gene of interest (7m8). rBV from each plaque and from each passage were coinfected with rBV comprising a stable gene of interest (GFP-Fluc) in insect cells to obtain rAAV. The rAAV titer obtained from coinfection with rBV comprising a stable gene of interest (GFP-Fluc) and rBV comprising 7m8 at the P0, P1 and P2 stage from each plaque is indicated in vg/ml. The AAV titers obtained with rBV from the PO, P1 and P2 stages were compared for each plaque and the ratio of titers is presented. The rAAV titers obtained from rBV comprising a stable gene of interest after P1 and P2 (plaque 2) are significantly higher than the rAAV titers from the P1 and P2 rBV from plaque 1.

FIG. 7 provides graphs summarizing rAAV titer results obtained in various experiments. The left graph summarizes rAAV titers obtained from insect cells grown the indicated media (Sf-900™ SFM, Sf-900™ II SFM, EX-CELL™ TiterHigh and ESF AF) in Sf-RVN cells with CFI/7m8. The center graph summarizes rAAV titers obtained from Sf-RVN cells grown in either ESF AF media or Sf-900™ II SFM media with and without a feed after co-infection. The rAAV titer obtained from ESF AF media was substantially higher than with Sf-900TM II SFM with or without feed. Addition of feed to the ESF-AF media increased the titer obtained. The right panel provides a graph summarizing results obtained from co-infection at a MOI of either 0.001 or 3 in ESF AF media or Sf-900™ II SFM media. Surprisingly the results obtained with MOI of 0.001 were significantly higher than titer obtained when a MOI of 3 was used in ESF AF media.

FIGS. 8A-C presents a summary of an evaluation of the level of donor plasmid (pFB) in three different recombinant bacmid preps. The level of the donor plasmid was evaluated by droplet digital (ddPCR) and the results are summarized in FIG. 8A. Bacmid prep 1 contains recombinant bacmids after counter-selection for the donor plasmid (Bacmid-GOI-no donor plasmid). The ratio of the gene of interest to DNApol was 1.19. The ratio of the donor plasmid to rBacmid was 0 (measured in fold). Bacmid prep 2 contains recombinant bacmids obtained using a donor plasmid comprising the ampicillin resistance gene (rather than a kanamycin resistance gene) without counter-selection for the donor plasmid (Bacmid-GOI-with donor plasmid (Amp)). The ratio of the gene of interest to DNApol was 5.45. The ratio of the donor plasmid to rBacmid was 4.6 fold. Bacmid prep 3 contains recombinant bacmids obtained using a donor plasmid comprising a kanamycin resistance gene (Kan), wherein the recombinant bacmid also comprises a kan resistance gene. The ratio of the gene of interest to DNApol was 309.18. The ratio of the donor plasmid to rBacmid was 261 fold. FIGS. 8B and 8C provide a graphical representation of the rBacmid copy number versus the donor plasmid comprising the gene of interest (pFB) copy number from the three Bacmid preps (FIG. 8B). The results from Bacmid Prep 3 (donor plasmid and bacmid plasmid with the same antibiotic resistance gene) are excluded from FIG. 8C.

### DETAILED DESCRIPTION OF THE INVENTION

As gene therapies are developed for use to treat common or prevalent diseases or disorders, the difficulties associated with large scale production of recombinant adeno-associated virus (rAAV) comprising a gene of interest have become apparent. There is a need in the art for efficient methods of producing large volumes of rAAV for clinical use. The application provides modified systems and methods for efficiently producing large amounts of rAAV. Further, the baculovirus expression vectors (rBV) used to produce rAAV exhibit a significant rate of loss of the gene of interest from the BEV. The current application provides rBV comprising a stable gene of interest, methods of obtaining rBV comprising a stable gene of interest and compositions and methods for obtaining recombinant bacmids and rBV comprising a stable gene of interest. Compositions comprising recombinant bacmids and rBV comprising a stable gene of interest may also be used as a master source or master viral bank (MVB). A master source or master viral bank may facilitate large scale clinical grade manufacturing, testing and regulatory approvals. Many approved gene therapies target rare or uncommon diseases or disorders. The amount of delivery vector needed for a rare or uncommon disease or disorder is limited. As gene therapies are developed for prevalent diseases or disorders, it will be necessary to produce much higher quantities of gene therapy delivery vectors such as rAAV. Prior to this work, limited methods of producing large quantities of rAAV existed. The prior methods were subject to challenges including but not limited to high contamination risk, substantial declines in the quality of rAAV produced over time, low efficiency of production and long production times.

Unless defined otherwise, all technical and scientific terms used herein have the meaning commonly understood by a person skilled in the art to which this invention belongs.

Numeric ranges are inclusive of the numbers defining the range. The term about is used herein to mean plus or minus ten percent (10%) of a value. For example, “about 100” refers to any number between 90 and 110. The term “about” a range refers to that range minus 10% of its lowest value and plus 10% of its greatest value. Reference to “about” a value or parameter herein includes (and describes) embodiments that are directed to that value or parameter per se.

As convention and as used throughout the application, a scientific format of exponential notation may be used, replacing part of the number with E+n, in which E (exponent) multiplies the preceding number by 10 to the nth power. For example, a 2-decimal scientific format displays 12345678901 as 1.23E+10, which is 1.23 times 10 to the 10th power, and may be written alternatively as 1.23×10¹⁰. Similarly, 1.23E−10 may be written alternatively as 1.23×10⁻¹⁰.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

The terminology used herein is for the purpose of describing particular examples only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing,”and is inclusive or open-ended.

By “consisting essentially of”, is intended a limitation of the scope of the, for example, composition, method, kit, etc., described to the specified materials that do not materially affect the basic and novel characteristic(s) of the, for example, composition, method, kit, etc. For example, an expression cassette “consisting essentially of” a coding sequence encoding a polynucleotide operably linked to a promoter and a polyadenylation sequence may include additional sequences, e.g., linker sequences so long as they do not materially affect the transcription or translation of the coding sequence. As another example, a variant or mutant polypeptide “consisting essentially of” a recited sequence has the amino acid sequence of the recited sequence plus or minus about 10 amino acid residues at the boundaries of the sequence based upon the full length naïve polypeptide from which it was derived, e.g. 10, 9, 8, 7, 6, 5, 4, 3, 2 or 1 residue less than the recited bounding amino acid residue or 1, 2, 3, 4, 5, 6, 7, 8, 9 or 10 residues more than the recited bounding amino acid residue.

Any reference to “or” herein is intended to encompass “and/or” unless otherwise stated.

Donor plasmids comprise a first antibiotic resistance gene, a counter selective marker and a transfer cassette, wherein the transfer cassette comprises a left transposon arm, a gene of interest and a right transposon arm. By “counter selective marker” is intended a selectable marker that eliminates or inhibits growth of the host organism upon selection. Counter selective markers are known in the art and include but are not limited to antibiotic sensitivity genes. Antibiotic sensitivity genes include, but are not limited to rpsl, a thymidine kinase gene and URA3. Donor plasmids for use in the systems and methods of the current application are suitable for use in bacterial cells.

AAV “rep” and “cap” genes refer to polynucleotide sequences encoding replication and encapsidation proteins of adeno-associated virus. AAV rep and cap are referred to herein as AAV “packaging genes.” In some embodiments, the AAV rep gene may be from any AAV serotype or may be a modified AAV rep gene. In some aspects, the AAV rep gene is of the same serotype as the ITRs of the rAAV vector genome. In some aspects, the AAV rep gene is of a different serotype than the ITRs of the rAAV vector genome or the Cap serotype. In some aspects, the AAV Rep is a chimeric Rep. In some aspects, the AAV Cap is a chimeric Cap.

AAV “rep” and “cap” genes refer to polynucleotide sequences encoding replication and encapsidation proteins of adeno-associated virus and variants thereof. The Rep genes encode the Rep proteins Rep78, Rep68, Rep52, and Rep40. The Rep79 and Rep68 proteins are multifunctional DNA binding proteins that perform helicase and nickase functions during productive replication to allow for the resolution of AAV termini (see e.g., Im et al 1990 Cell 61:447-57. These proteins also regulate transcription from endogenous AAV promoters and promoters within helper viruses (see e.g., Periera et al (1997) J. Virol 71:1079-1088). The other Rep proteins modify the function of Rep78 and Rep68. “Rep”encompasses variant Rep, chimeric Rep, and modified Rep.

The Cap genes encode the capsid proteins VP1, VP2 and VP3. Cap encompasses variant Cap, chimeric Cap, pseudotyped Cap and modified Cap. One skilled in the art would be aware of which Rep and Cap forms are preferred for use together and would select appropriately to form Rep/Cap. The term Rep/Cap refers to a nucleotide sequence encoding functional forms of Rep and Cap. In some embodiments, the polynucleotide comprising the polynucleotide encoding Cap further comprises a polynucleotide encoding Rep.

AAV Rep and ITR sequences efficiently cross-complement other AAV Rep and ITR sequences in insect cells. Generally, the Cap proteins, which determine the cellular tropicity of the rAAV particle and related Cap protein-encoding sequences are significantly less conserved than Rep proteins and genes among different AAV serotypes. In view of the ability of Rep and ITR sequences to cross-complement corresponding sequences of other serotypes, pseudotyped AAV particles comprising the capsid proteins of a serotype (for example AAV6) and the Rep and/or ITR sequences of another AAV serotype (for example AAV2) can be readily generated. As used herein, “pseudo-typed” refers to the source of the Cap protein in an adeno-associated virus. See Halbert et al 2000 J. Virol. 74:1524-1532 and Halbert et al 2001 J. Virol 75:6615-6624. rAAV2/6 and rAAV2/8 (that is pseudotyped AAV comprising the ITRs and Rep sequences of AAV2 and VP sequences derived from AAV6 and AAV8 respectively in insect cells. Production of pseudotyped AAV vector comprising the Cap genes of a particular AAV serotype indicate that non-pseudotyped vectors of that serotype can be successfully produced in that system.

Sequences from more than one AAV serotype can be combined for production of AAV in insect cells. For example, a nucleic acid comprising at least one AAV ITR nucleotide sequence can be derived from one serotype while other nucleic acids can comprise open reading frames or coding sequences derived from one or more other serotypes. The nucleic acids of any of AAV serotypes 1, 2, 3, 4, 5, 6, 7, 8, 9, 10 and 11 can provide a Rep gene, a Cap gene, and/or an AAV ITR in the present methods.

In some aspects of the methods, an AAV ITR can be AAV1, aAV2 or an AAV6 ITR; a nucleic acid comprising the Rep ORFs can comprise an AAV1, an AAV2 or an AAV6 Rep gene and a nucleic acid comprising the Cap ORFs can comprise an AAV1, an AAV2 or an AAV6 Cap gene. Modified AAV sequences may also be used to produce rAAV in insect cells. Nucleotide sequence having at least about 70%, at least about 80%, at least about 90%, at least about 95% sequence identity to an AAV1, AAV2, AAV3 and/or AAV4 sequence may be used in place of wild-type AAV Rep, AAV-ITR or AAV-Cap sequences provided that rAAV particles are produced in infected cells.

In some embodiments, the one or more ITRs are AAV1, AAV2, AAV3, AAV4, AAV5, AAV6, AAV7, AAV8, AAV9, AAV10, avian AAV, bovine AAV, canine AAV, equine AAV, primate AAV, non-primate AAV, rhesus macaque AAV, or ovine AAV ITR's or variants thereof. In some embodiments, the one or more ITRs are AAV1,AAV2, AAV3, AAV4, AAV5, AAV6, AAV7, AAV8, AAV9, AAV10, avian AAV, bovine AAV, canine AAV, equine AAV, primate AAV, non-primate AAV, rhesus macaque AAV, or ovine AAV ITR's. In some embodiments, the one or more ITRs are AAV1, AAV2, AAV3, AAV4, AAV5, AAV6, AAV7, AAV8, AAV9, AAV10, avian AAV, bovine AAV, canine AAV, equine AAV, primate AAV, non-primate AAV, rhesus macaque AAV, or ovine AAV ITR comprising one or more insertions, deletions and/or substitutions of nucleotides.

Any vector known in the art may be employed with the present teachings provided it is insect cell-compatible. The presence of a vector in the insect cell need not be permanent. The vectors may be introduced by any method known, for example by chemical treatment of cells, electroporation or infection. A “vector” as used herein refers to a macromolecule or association of macromolecules that comprises or associates with a polynucleotide and which can be used to mediate delivery of the polynucleotide to a cell. Illustrative vectors include, but are not limited to, plasmids, viral vectors (i.e., adeno-associated viruses), liposomes and other gene delivery vehicles, and bacmids.

An “AAV vector” or “rAAV vector” as used herein refers to an adeno-associated virus (AAV) vector or a recombinant AAV (rAAV) vector comprising a polynucleotide sequence not of AAV origin (e.g., a polynucleotide heterologous to AAV such as a nucleic acid sequence that encodes a therapeutic transgene, e.g., human complement factor inhibitor (CFI) for transduction into a target cell or to a target tissue. In general, the heterologous polynucleotide is flanked generally by two AAV inverted terminal repeat sequences (ITRs). The term rAAV vector encompasses both rAAV vector particles and rAAV vector plasmids. A rAAV vector may be either single-stranded (ssAAV) or self-complementary (scAAV).

An “AAV virus” or “AAV viral particle” or “rAAV vector particle” or “rAAV particle” refers to a viral particle comprising at least one AAV capsid protein and a polynucleotide rAAV vector. In some cases, the at least one AAV capsid protein is from a wild type AAV or is a variant AAV capsid protein. By “variant AAV capsid protein” is intended that the AAV capsid protein comprises at least one amino acid difference (e.g., amino acid substitution, amino acid insertion, amino acid deletion) relative to a corresponding parental AAV capsid protein. The variant capsid protein may confer increased infectivity of a retinal cell as compared to the infectivity of a retinal cell by an AAV virion comprising an amino acid sequence present in a naturally occurring AAV capsid protein. Variant AAV capsid proteins may include, but are not limited to, an AAV capsid protein with an insertion, an insertion of the 7m8 amino sequence, an R100 insertion, a 7m8 like insertion, an LSV1 sequence replacement and any other engineered capsid protein generated by other strategies (e.g., DNA shuffling, directed evolution, peptide insertion, ancestral reconstruction, among others). The LSV1 replacement sequence and the 7m8 insertion sequence are known in the art (see, for example, U.S. Pat. Nos. 9,193,956; 9,233,133; U.S. Pub. No. US2021/0040501; and PCT/US2020/029895). Variant AAVs of particular interest may include, but are not limited to, those disclosed in U.S. Pat. No. 9,193,956, WO2017197355, WO2018022905, WO2019104279 and/or US20210371879A1. In some embodiments, the variant AAV comprises or consists of the 7m8 variant capsid protein (which may be referred to as AAV2.7m8 and 7m8AAV2). In some embodiments, the AAV comprises or consists of an AAV2.5T capsid protein such as provided in U.S. Pat. No. 9,233,131. In some embodiments, the AAV comprises the AAVShH10 or AAV6 capsid protein (U.S. Patent Application Pub. No. 20120164106 and Klimczak et al PLOS One 4(10): e7467 (Oct. 14, 2009). In some embodiments, the AAV comprises or consists of an AAV2.5T_LSV1 variant disclosed in U.S. Patent App Pub. No. WO2020219933.

The AAV2.7m8 capsid was engineered from wild-type AAV2 and exhibits highly efficient retinal transduction following intravitreal (IVT) injection. The AAV2.7m8 capsid has an insertion of 7m8 in loop IV, reduces interactions between the virus capsid and the HSPG receptor and allows diffusion across the inner limiting membrane (ILM) into the retina. AAV2.7m8 can transduce retinal cells including photoreceptors, Muller glial cells, retinal ganglion cells, bipolar cells, and RPE cells.

If the particle comprises a heterologous polynucleotide (e.g., a polynucleotide other than a wild-type AAV genome such as a transgene to be delivered to a target cell or target tissue), it is referred to as a “rAAV particle”, “rAAV vector particle” or a “rAAV vector”. Thus, production of rAAV particles necessarily includes production of a rAAV vector, as such a vector contained within a rAAV particle. In general, the heterologous polynucleotide is flanked by AAV inverted terminal repeat sequences (ITRs). A heterologous polynucleotide may comprise a polynucleotide cassette. A polynucleotide cassette of the present application can be packaged in a variant AAV particle to promote delivery of the cassette to a cell type of interest such as, but not limited to a retinal cell, in a target tissue.

The term “packaging” as used herein can refer to a series of intracellular events that can result in the assembly and encapsidation of a rAAV particle.

By “passage” is intended a process comprising the steps of infecting insect cells with rBV comprising a gene of interest and incubating the infected insect cells.

The terms “gene of interest”, “GOI” and “transgene” are used interchangeably herein. A gene of interest comprises an open reading frame encoding a gene product of interest. In various aspects, a nucleic acid may comprise two or more nucleic acid sequences each comprising a gene of interest encoding a gene product. A “gene product” is a molecule resulting from expression of a particular gene. Gene products include, but are not limited to, a polypeptide, an aptamer, an interfering RNA, an mRNA, and the like. In particular embodiments, a “gene product” is a polypeptide, peptide, protein or interfering RNA including short interfering RNA (siRNA), miRNA or small, hairpin RNA (shRNA). In some embodiments, the gene of interest may be a reporter gene.

Reporter genes are known in the art and include, but are not limited to, chloramphenicol acetyl transferase, a β-galactosidase, a β-glucoronidase, a renilla luciferase, a firefly luciferase, a green fluorescent protein (GFP), a red fluorescent protein (RFP) and an alkaline phosphatase such as secreted alkaline phosphatase. In some embodiments, the gene of interest may encode one or more AAV proteins or polypeptides. AAV proteins or polypeptides may include, but are not limited to Rep proteins, Rep78, Rep 68, Rep58, Rep40, Cap proteins, VP1, VP2, VP3 and fragments and variants thereof, including but not limited to Rep/Cap. In some embodiments, the gene of interest may encode a reporter gene product such as, but not limited to, GFP and RFP. In some embodiments, the gene of interest may encode a therapeutic gene product such as a therapeutic protein. Therapeutic gene products are known in the art and include, but are not limited to, a polypeptide hormone, cytokine or growth factor (e.g. insulin or erythropoietin), an interferon, a blood clotting factor, a vaccine, an anti-angiogenic polypeptide, a vascular endothelial growth factor (VEGF) binding protein, an anti-VEGF agent, an anti-VEGF protein, an opsin protein, an anti-C3 antibody, an anti-C5 antibody, a hormone receptor (such as but not limited to mineral corticosteroid, glucocorticoid, and thyroid hormone receptors), intramembrane proteins (such as but not limited to TM-1 and TM-7), intracellular receptors (such as but not limited to orphans, retinoids, vitamin D3 and vitamin A receptors), signaling molecules (such as but not limited to kinases, transcription factors and signal transducers and activators of transcription receptors of the cytokine superfamily (e.g. erythropoietin, growth hormone, interferons, interleukins and colony stimulating factors); G-protein coupled receptors such as but not limited to hormones, calcitonin, epinephrine, gastrin, paracrine or autocrine mediators such as somatostatin or prostaglandins; neurotransmitter receptors (norepinephrine, dopamine, serotonin or acetylcholine); ligands of tyrosine kinase receptors such as insulin growth factor and nerve growth factor; and an anti-dry AMD gene product. Anti-VEGF agents are known in the art and include, but are not limited to, bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept.

The term “anti-VEGF agent” includes any therapeutic agent, including proteins, polypeptides, peptides, fusion protein, multimeric proteins, gene products, antibody, human monoclonal antibody, antibody fragment, aptamer, small molecule, kinase inhibitor, receptor or receptor fragment, or nucleic acid molecule, that can reduce, interfere with, disrupt, block and/or inhibit the activity or function of an endogenous VEGF and/or an endogenous VEGF receptor (VEGFR), or the VEGF-VEGFR interaction or pathway in vivo. An anti-VEGF agent can be any one of the known therapeutic agents that can reduce new blood vessel growth or formation and/or edema, or swelling, when delivered into a cell, tissue, or a subject in vivo, e.g., ranibizumab, brolucizumab, or bevacizumab. In some embodiments, an anti-VEGF agent can be naturally occurring, non-naturally occurring, or synthetic. In some embodiments, an anti-VEGF agent can be derived from a naturally occurring molecule that was subsequently modified or mutated to confer an anti-VEGF activity. In some embodiments, an anti-VEGF agent is a fusion or chimeric protein. In such proteins, functional domains or polypeptides are artificially fused to a moiety or a polypeptide to make a fusion or chimeric protein that can sequester VEGF in vivo or function as a VEGFR decoy. In some embodiments, an anti-VEGF agent is a fusion or chimeric protein that blocks endogenous VEGFR from interacting with its ligands.

As used herein, “VEGF” can refer to any isoform of VEGF, unless required otherwise, including, but not limited to, VEGF-A, VEGF-B, VEGF-C, VEGF-D, VEGF-E, VEGF-F, or any combination, or any functional fragment or variant thereof. Unless required otherwise, “VEGF” can refer to any member of the VEGF family, including members: VEGF-A, placenta growth factor (PGF), VEGF-B, VEGF-C, and VEGF-D, or any combination, functional fragment, or variant thereof. As used herein, “VEGF receptor” or “VEGFR” or “VEGF-R” can be used to refer to any one of the receptors of VEGF, including, but not limited to, VEGFR-1 (or Flt-1), VEGFR-2 (or Flk-1/KDR), and VEGFR-3 (or Flt-4). VEGFR can be a membrane bound or soluble form, or a functional fragment or truncation of a receptor.

Therapeutic gene products for use in treating an ocular disease or disorder may include, but are not limited to, an anti-angiogenic polypeptide, a VEGF binding protein, an opsin protein, an anti-C3 antibody, an anti-C5 antibody, an anti-dry AMD gene product, aflibercept, sFLT-1, CFI, ranibizumab and bevacizumab. An anti-dry AMD gene product may include, but is not limited to, inhibitors of C3, C5, HtrA1, C1qm, and natural inhibitors of the complement pathway, such as CFI, CFH and CD59.

As used herein, a “therapeutic gene” refers to a gene that, when expressed produces a therapeutic gene product that confers a beneficial effect on the cell or tissue when it is present or on a mammal in which the gene is expressed. Examples of beneficial effects include amelioration of a sign or symptom of a condition or disease, prevention or inhibition of a condition or disease, or conferral of a desired characteristic. Therapeutic genes include, but are not limited to, genes that corrects a genetic deficiency in a cell or mammal and genes that express a therapeutic gene product.

“Helper function(s)” refer to function(s) encoded in a helper virus genome which allow AAV replication and packaging (in conjunction with other requirements for replication and packaging). As described herein, “helper function” may be provided in a number of ways including but not limited to providing helper virus or providing for example a helper plasmid comprising polynucleotide sequences encoding the requisite functions to a producer cell in trans. Requisite functions include but are not limited to the functions provided by the Adeno VA, E4 and E2A genes. For example, a plasmid or other expression vector comprising nucleotide sequences encoding one or more adenoviral helper proteins may be transfected or co-transfected into a producer cell.

By “plasmid” is intended a small often circular extrachromosomal DNA molecule that can replicate independently in a cell. A plasmid may comprise one or more expression cassettes, open reading frames, polynucleotide cassettes, or expression vectors. A helper plasmid of the present system provides a helper function and comprises an antibiotic resistance gene. In some instances, multiple copies of a helper plasmid are present in a bacterial cell comprising a donor plasmid and a bacmid. An increased helper plasmid copy number may be beneficial in methods of obtaining a recombinant bacmid or BEV comprising a stable gene of interest.

Antibiotic resistance genes are known in the art and include, but are not limited to ampicillin resistance genes, chloramphenicol resistance genes, erythromycin resistance genes, gentamycin resistance genes, kanamycin resistance genes, penicillin resistance genes, streptomycin resistance genes, penicillin/streptomycin resistance genes, rifampicin resistance genes, and tetracycline resistance genes. See, for example, Martinez et al 2008 Science 321:365-367 and Pal et al 2014 Nuc. Acids Res. 42:D737-43. One skilled in the art is capable of selecting antibiotic resistance genes suitable for use in the claimed methods.

As used herein, “VEGF” can refer to any isoform of VEGF, unless required otherwise, including, but not limited to, VEGF-A, VEGF-B, VEGF-C, VEGF-D, VEGF-E, VEGF-F, or any combination, or any functional fragment or variant thereof. Unless required otherwise, “VEGF” can refer to any member of the VEGF family, including members: VEGF-A, placenta growth factor (PGF), VEGF-B, VEGF-C, and VEGF-D, or any combination, functional fragment, or variant thereof. As used herein, “VEGF receptor” or “VEGFR” or “VEGF-R” can be used to refer to any one of the receptors of VEGF, including, but not limited to, VEGFR-1 (or Flt-1), VEGFR-2 (or Flk-1/KDR), and VEGFR-3 (or Flt-4). VEGFR can be a membrane bound or soluble form, or a functional fragment or truncation of a receptor. The modified Bac-to-Bac systems of the present application make use of a first antibiotic resistance gene, a second antibiotic resistance gene and a third antibiotic resistance gene. One skilled in the art may select any three separate antibiotic resistance genes for use in the modified Bac-to-Bac system as long as each of the three antibiotic resistance genes selected confer resistance to a different antibiotic and the antibiotic resistance gene of the donor plasmid does not interfere with the counter selective marker utilized in the system. In various embodiments, the antibiotic resistance gene of the donor plasmid is designated as a first antibiotic resistance gene, the antibiotic resistance gene of the helper plasmid is designated as a second antibiotic resistance gene and the antibiotic resistance gene of the bacmid is designated as a third antibiotic resistance gene. In an embodiment, the first antibiotic resistance gene, the second antibiotic resistance gene and the third antibiotic resistance gene are selected from the group comprising ampicillin resistance genes, tetracycline resistance gene and kanamycin resistance genes. In some aspects, of the method, the first antibiotic resistance gene is an ampicillin resistance gene, the second antibiotic resistance gene is a tetracycline resistance gene and the third antibiotic resistance gene is a kanamycin resistance gene.

In some embodiments the baculovirus expression vector comprises a gene of interest encoding Rep/Cap operably linked to a promoter sequence that drives expression of the polynucleotide in a cell. In some embodiments the baculovirus expression vector comprises a gene of interest encoding a therapeutic gene product operably linked to a promoter sequence that drives expression of the polynucleotide in a cell. In certain embodiments, the cell is a production cell. By “production cell” is intended a host cell used to produce rAAV virions. Exemplary host cells include insect cells including, but not limited to, Sf-9 cells, Sf-21 cells, Sf-RVN, Drosophila cell lines, Hi-5 cells and cell lines derived from Aedes albopictus. The production cell may be used to produce virions comprising Rep/Cap and a gene of interest. In certain embodiments, the cell is a subject cell. By “subject cell” is intended the cell of a subject which has received rAAV gene therapy. It is recognized that different promoters may be utilized to drive expression in a production cell rather than in a subject cell. For example, a gene of interest encoding Rep/Cap may be operably linked to an insect-operable promoter. Insect-operable promoters include, but are not limited to, the polyhedron promoter (polh). A gene of interest encoding a therapeutic gene product may be operably linked to a promoter sequence that is operable in a subject cell. The gene of interest may be operably linked to one or more regulatory regions that impact expression in a subject cell.

A bacmid is a baculovirus plasmid that can be propagated in both bacterial cells and insect cells; in some instances, a bacmid may be considered a shuttle vector.

The terms “rBVs”, “baculovirus expression vectors”, and “BEVs” are used interchangeably herein. A baculovirus expression vector is a eukaryotic DNA viral vector for cloning and/or expression in cultured lepidopteran insect cells or insects. Most baculovirus expression vector are derived from the Autographa californica nuclear polyhedrosis virus (AcNPV). Methods of making and using rBV are known in the art. See for example Chambers et al 2018 Curr Protoc Prot Sci 91:5.4.1-5.4.6 and Felberbaum 2015 Biotechnol. 10:702-714.

A “promoter” is a region of DNA that initiates transcription of a particular gene. Promoters from a wide variety of sources are well known in the art, and any promoter known in the art may be utilized in the methods and systems of the application. Promoters may be unidirectional (i.e., initiate transcription in one direction) or bi-directional (i.e., initiate transcription in either a 3′ or 5′ direction). Promoters include, but are not limited to, constitutively active promoters, inducible promoters and cell-type specific promoters. Constitutively active promoters include, but are not limited to, human beta-actin, chicken beta-actin, cytomegalovirus (CMV), SV40 and the CAG promoter. Cell-type specific promoters include but are not limited to the CD19 gene promoter, CaMKII1 and UAS. Inducible promoters include, but are not limited to the Tet system (U.S. Pat. Nos. 5,464,758 and 5,814,618), the ecdysone inducible system (No et al, (1996) Proc. Natl Acad Sci 93:3346-3351, the Cre-Lox system, the T-Rex™ system (Invitrogen, Carlsbad CA), the Cre-ERT tamoxifen inducible recombinase system (Indra et al (1999) Nuc Acid Res 27:4324-4327, U.S. Pat. No. 7,112,715, Kramer & Fussenegger 2005 Methods Mol Biol 308:123-144 and the LacSwitch™ system (Stratagene San Diego CA). Insect operable promoters include, but are not limited to polh, IE-1, IE-0, IE-2, 39K, gp64, p6.9, VLf-1, p10, p10-p6.9, pCap/polh, promoter, the ORF46 promoter, polh-L21, hsp70, Mtn promoter, pTre-CMV, pB2, pB2-p10, GAPDH promoter, OPie2, hr5-ie1-p10 promoter.

Transposon insertion sites are known in the art and include, but are not limited to, Tn7, mini-att-Tn7, loxP, attR1/attR2.

“Multiplicity of Infection” or “MOI” refers to the number of virions or virus particles per cell during an infection. A “low MOI” is an MOI below about 2, below about 1, below about 0.1, below about 0.01, below about 0.009, below about 0.008, below about 0.007, below about 0.006, below about 0.005, below about 0.004, below about 0.003, below about 0.002, about 0.001, about 0.0005 or about 0.0001. A low MOI may be in the range of about 0.0005 to about 0.09, about 0.0008 to about 0.05, about 0.001 to about 0.04, about 0.001 to about 0.03, about 0.001 to about 0.02, about 0.001 to about 0.01, about 0.001 to about 0.009, about 0.001 to about 0.008, about 0.001 to about 0.007, about 0.001 to about 0.006, about 0.001 to about 0.005, about 0.001 to about 0.004, about 0.001 to about 0.003, and about 0.001 to about 0.002. MOIs suitable for use with rBV and insect cells may differ substantially from MOIs suitable for use with 10^{about} 2.3, 3, 4, 5, 6, 7, 8, 9, 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷ and 1×10¹⁸.

The terms “harvested” and “collected” may be used interchangeably. Methods of harvesting virions, viral particles and rAAV are known in the art and may include, but are not limited to filtration, flow filtration, depth filtration, membrane filtration, centrifugation, and combinations thereof. Any method of harvesting virus particles known in the art may be used in the methods and systems of the application. The amount of virus particles harvested from insect cells in the various embodiments of the method may be in the range of about 1×10¹⁰ vg, 5×10¹⁰ vg, 6×10¹⁰ vg, 7×10¹⁰ vg, 8×10¹⁰ vg, 9×10¹⁰ vg, 1×10¹¹ vg, 2×10¹¹ vg, 3×10¹¹ vg, 4×10¹¹ vg, 5×10¹¹ vg, 6×10¹¹ vg, 7×10¹¹ vg, 8×10¹¹ vg, 9×10¹¹ vg, 1×10¹² vg, 2×10¹², 3×10¹², 4×10¹² vg, 5×10¹² vg, 6×10¹² vg, 7×10¹² vg, 8×10¹² vg, 9×10¹² vg, 1×10¹³ vg, 2×10¹³ vg, 3×10¹³ vg, 4×10¹³ vg, 5×10¹³ vg, 6×10¹³ vg, 7×10¹³ vg, 8×10¹³ vg, 9×10¹³ vg, 1×10¹⁴ vg, 2 x 10¹⁴ vg, 3×10¹⁴ vg, 4×10¹⁴ vg, 5×10¹⁴ vg, 6×10¹⁴ vg, 7×10¹⁴ vg, 8×10¹⁴ vg, 9 x 10¹⁴ vg, 1×10¹⁵ vg, 2×10¹⁵ vg, 3×10¹⁵ vg, 4×10¹⁵ vg, 5×10¹⁵ vg, 6×10¹⁵ vg, 7 x 10¹⁵ vg, 8×10¹⁵ vg, 9×10¹⁵ vg, 1×10¹⁶ vg, 2×10¹⁶ vg, 3×10¹⁶ vg, 4×10¹⁶ vg, 5 x 10¹⁶ vg, 6×10¹⁶ vg, 7×10¹⁶ vg, 8×10¹⁶ vg and about 9×10¹⁶ vg. It is recognized that the methods of harvesting rBV used in the methods may be the same or different from the methods of harvesting rAAV used in the methods.

The amount of rBV harvested from insect cells in the various embodiments of the method may be in the range of about 1×10¹⁰ vg, 5×10¹⁰ vg, 6×10¹⁰ vg, 7×10¹⁰ vg, 8×10¹⁰ vg, 9×10¹⁰ vg, 1×10¹¹ vg, 2×10¹¹ vg, 3×10¹¹ vg, 4×10¹¹ vg, 5×10¹¹ vg, 6×10¹¹ vg, 7×10¹¹ vg, 8×10¹¹ vg, 9×10¹¹ vg, 1×10¹² vg, 2×10¹², 3×10^{12,} 4×10¹² vg, 5×10¹² vg, 6×10¹² vg, 7×10¹² vg, 8×10¹² vg, 9×10¹² vg, 1×10¹³ vg, 2×10¹³ vg, 3×10¹³ vg, 4×10¹³ vg, 5×10¹³ vg. A recombinant baculovirus seed stock produced by the present methods may be in the range of about 1×10⁸ vg/ml, 5×10⁸ vg/ml, 1×10⁹ vg/ml, 2×10⁹ vg/ml, 3×10⁹ vg/ml, 4×10⁹ vg/ml, 5×10⁹ vg/ml, 6×10⁹ vg/ml, 7×10⁹ vg/ml, 8×10⁹ vg/ml, 9×10⁹ vg/ml, 1×10¹⁰ vg/ml, 2×10¹⁰ vg/ml, 3×10¹⁰ vg/ml, 4×10¹⁰ vg/ml, 5×10¹⁰ vg/ml, 6×10¹⁰ vg/ml, 7×10¹⁰ vg/ml, 8×10¹⁰ vg/ml, 9×10¹⁰ vg/ml, 1×10¹¹ vg/ml, 2×10¹¹ vg/ml, 3×10¹¹ vg/ml, 4×10¹¹ vg/ml, 5×11¹¹ vg/ml, 6×10¹¹ vg/ml, 7×10¹¹ vg/ml, 8×10¹¹ vg/ml, 9×10¹¹ vg/ml or about 1×10¹² vg/ml.

The amount of AAV viral particles harvested from insect cells in the various methods may be in the range of about 1×10¹¹ vg, 2×10¹¹ vg, 3×10¹¹ vg, 4×10¹¹ vg, 5×10¹¹ vg, 6×10¹¹ vg, 7×10¹¹ vg, 8×10¹¹ vg, 9×10¹¹ vg, 1×10¹² vg, 2×10¹², 3×10¹², 4×10¹² vg, 5×10¹² vg, 6×10¹² vg, 7×10¹² vg, 8×10¹² vg, 9×10¹² vg, 1×10¹³ vg, 2×10¹³ vg, 3×10¹³ vg, 4×10¹³ vg, 5×10¹³ vg, 6×10¹³ vg, 7×10¹³ vg, 8×10¹³ vg, 9×10¹³ vg, 1×10¹⁴ vg, 2×10¹⁴ vg, 3×10¹⁴ vg, 4×10¹⁴ vg, 5×10¹⁴ vg, 6×10¹⁴ vg, 7×10¹⁴ vg, 8×10¹⁴ vg, 9×10¹⁴ vg, 1×10¹⁵ vg, 2×10¹⁵ vg, 3×10¹⁵ vg, 4×10¹⁵ vg, 5×10¹⁵ vg, 6×10¹⁵ vg, 7×10¹⁵ vg, 8×10¹⁵ vg, 9×10¹⁵ vg, 1×10¹⁶ vg, 2×10¹⁶ vg, 3×10¹⁶ vg, 4×10¹⁶ vg, 5×10¹⁶ vg, 6×10¹⁶ vg, 7×10¹⁶ vg, 8×10¹⁶ vg and about 9×10¹⁶ vg. The titer of the harvested rAAV may be at least about 111 vg/ml, 211 vg/ml, 311 vg/ml, 411 vg/ml, 511 vg/ml, 611 vg/ml, 711 vg/ml, 811 vg/ml, 911 vg/ml, at least 112 vg/ml, 212 vg/ml, 312 vg/ml, 412 vg/ml, 512 vg/ml, 612 vg/ml, 712 vg/ml, 812 vg/ml, 912 vg/ml, at least 113 vg/ml, at least 114 vg/ml, or at least about 115 vg/ml.

By “polynucleotide cassette” is meant a polynucleotide sequence comprising two or more functional polynucleotide sequences, e.g., regulatory elements, translation initiation sequences, coding sequences, termination sequences, etc. typically in operable linkage to at least one other functional polynucleotide sequence in the polynucleotide cassette. Generally, a subject polynucleotide cassette is composed of DNA.

The polynucleotide cassettes of the present disclosure typically comprise a promoter region. A “promoter” as used herein encompasses a DNA sequence that directs the binding of RNA polymerase and thereby promotes RNA synthesis. Promoters and corresponding protein or polypeptide expression may be ubiquitous, meaning strongly active in a wide range of cells, tissues and species, or cell-type specific, tissue-specific or species specific. Promoters may be “constitutive”, meaning continuously active or “inducible” meaning the promoter can be activated or deactivated by the presence or absence of biotic or abiotic factors. In certain embodiments, the promoter region promotes expression of the coding sequence in mammalian cells. Suitable examples include the actin, chicken β-actin (CBA), cytomegalovirus (CMV), CMV immediate enhancer/β-actin (CAG), elongation factor 1 alpha (EF1a), and glyceraldehyde 3-phosphate dehydrogenase (GAPDH) promoters. A promoter may show tissue or cell specific expression.

As used herein, the term “operably linked” refers to a juxtaposition of genetic elements, e.g., promoter, enhancer, termination signal sequence, polyadenylation sequence, Kozak sequence, etc., wherein the elements are in a relationship permitting them to operate in the expected manner. For instance, a promoter is operably linked to a coding region if the promoter helps initiate transcription of the coding sequence. There may be intervening residues between the promoter and the coding sequence or between any two elements so long as the functional relationship is maintained.

Any concentration of viral particles suitable to effectively transduce mammalian cells can be prepared for contacting mammalian cells in vitro or in vivo. For example, the viral particles may be formulated at a concentration of 10⁸ vector genomes per mL (vg/ml) or more, for example, 5×10⁸ vg/mL; 10⁹ vg/mL, for example, 5×10⁹ vg/ml; 10¹⁰ vg/ml, for example 5×10¹⁰ vg/ml; 10¹¹ vg/ml, for example 5×10¹¹ vg/ml; 10¹² vg/ml, for example 5×10¹² vg/ml; 10¹³ vg/ml, for example 5×10¹³ vg/ml; 10¹³ vg/ml, for example 1.5×10¹³ vg/ml; 10¹⁴ vg/ml, for example 1×10¹⁴ vg/ml and 5×10¹⁴ vg/ml or more, but typically not more than 1×10¹⁵ vg/ml. Similarly, any total number of viral particles suitable to provide appropriate transduction of cells to confer the desired effect or treat the disease can be administered to the mammal.

The subject viral vector may be formulated into a pharmaceutical composition comprising any suitable unit dose of the vector which can be administered to a subject to produce a change in the subject or to treat a disease in the subject. In some embodiments, a unit dose comprises, without limitation, 1×10⁸ vg or more, for example at least about 1×10⁹ vg, 1×10¹⁰ vg, 1×10¹¹ vg, 1×10¹² vg, 1×10¹³ vg, 1×10¹⁴ vg or 1×10¹⁵ vg. In some embodiments a unit dose is from about 1×10⁹ to about 4×10¹² vg, from about 1×10¹⁰ to about 4×10¹¹ vg, from about 2×10¹⁰ to about 3×10¹¹ vg, from about 2×10¹⁰ to about 2×10¹¹ vg, from about 2.5 X 10¹⁰ to about 2×10¹¹ vg, from about 2×10¹⁰ to about 1×10¹¹ vg, from about 5×10⁹ to about 8×10¹¹ vg, from about 1×10¹⁰ to about 2×10¹¹ vg, from about 5×10¹⁰ to about 2×10¹¹ vg, or from about 8×10¹⁰ to about 1×10¹¹ vg.

A previously unsolved problem in the field of rAAV production from baculovirus expression vectors is loss of the gene of interest from the rBV. Compositions and methods for obtaining baculovirus expression vectors that exhibit a reduced rate of loss of the gene of interest are provided herein. The methods and compositions allow production of a recombinant baculovirus seed stock comprising a gene of interest.

Methods of producing a recombinant baculovirus seed stock comprising a gene of interest, wherein at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95% of the rBV in the recombinant baculovirus seed stock comprise the gene of interest are provided herein. In some instances, a recombinant baculovirus seed stock comprising rBVs comprising a stable gene of interest may be re-plaqued and regenerated. In some instances, recombinant baculovirus seed stock comprising rBV's comprising a stable gene of interest may be stored. Storage may involve re-passaging the recombinant baculovirus seed stock. Thus, a recombinant baculovirus seed stock comprising rBV comprising a stable gene of interest may be used as master viral bank, a starting viral bank, a regenerative bank or an original supply.

rBV of the present application may exhibit a low rate of loss of the gene of interest. By a low rate of loss of the gene of interest is intended a loss of the gene of interest from less than about 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78% or less than about 80% of the rBV per passage.

By “stable gene of interest” is intended the gene of interest is maintained within a vector at a high retention rate through one or more cycles through cells. The vector may be a recombinant bacmid and the cells may be either bacterial cells or insect cells. The vector may be a recombinant BEV and the cells may be insect cells. By a cycle through a cell is intended introduction of the vector to a cell and harvest of vectors from a cell or cells. A cycle through a cell may involve additional steps including but not limited to a purification step or a concentration step. It is recognized that the retention rate of a gene of interest in a bacmid may be the same as or different from the retention rate of the same gene of interest construct in a BEV. It is recognized that the retention rate of a gene of interest construct in rBV may be higher than the retention rate of the same gene of interest construct in a recombinant bacmid. It is recognized that the retention rate of a gene of interest construct in rBV may be lower than the retention rate of the same gene of interest construct in a recombinant bacmid. The retention rate is the ratio of the gene of interest to the vector in a population of vectors.

The retention rate of a gene of interest in a vector may range from 0 to 1. rBV exhibit retention rates of 0, about 0.001, about 0.005, about 0.01, about 0.05, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, and about 1. High retention rates include a retention rate above about 0.5,above about 0.55, above about 0.6, above about 0.65, above about 0.7, above about 0.75, above about 0.8, above about 0.85, above about 0.9, above about 0.95, and about 1 after passage 1 (or after cycle 1); a retention rate above about 0.4, above about 0.45, above about 0.5, above about 0.55, above about 0.6, above about 0.65, above about 0.7, above about 0.75, above about 0.8, above about 0.85, above about 0.9, above about 0.95, and about 1 after passage 2 (or after cycle 2); and a retention rate above about 0.35 above about 0.4, above about 0.45, above about 0.5, above about 0.55, above about 0.6, above about 0.65, above about 0.7, above about 0.75, above about 0.8, above about 0.85, above about 0.9, above about 0.95, and about 1 after passage 3 (or after cycle 3).

A stable gene of interest may exhibit a gene of interest to vector ratio equal to or greater than about 0.5, about 0.55, about 0.6, about 0.65, about 0.7, about 0.75, about 0.8, about 0.85, about 0.9, about 0.95, and about 1 after Passage 1; equal to or greater than about 0.4, about 0.45, about 0.5, bout 0.55, about 0.6, about 0.65, about 0.7, about 0.75, about 0.8, about 0.85, about 0.9, about 0.95, and about 1 after Passage 2; or equal to or greater than about 0.35, about 0.4, about 0.45, about 0.5, bout 0.55, about 0.6, about 0.65, about 0.7, about 0.75, about 0.8, about 0.85, about 0.9, about 0.95, and about 1 after three or more passages. It is recognized that the presence of donor plasmid comprising the gene of interest may artificially increase the gene of interest to vector ratio.

Vector level may be evaluated by evaluating any suitable portion of the vector including but not limited to, a promoter region and a backbone region, particularly preferred portions of the vector are those regions unique to the bacmid plasmid.

Compositions and methods of producing a vector comprising a stable gene of interest are provided herein. While not being bound by mechanism, a stable gene of interest may result from a process involving concatemer formation, increased transposase levels, altered integration, or a combination of any of those elements. Maintaining selection for the helper plasmid up to and including during the process of counter selection for the donor plasmid may be beneficial for producing vectors comprising a stable gene of interest. Increasing production of transposase may be beneficial for formation of vectors comprising a stable gene of interest.

In some cases, the unit dose of a pharmaceutical composition may be measured using multiplicity of infection (MOI). By MOI it is meant the ratio or multiple of vector or viral genomes to the cells to which the nucleic acid may be delivered. In some cases, the MOI may be 1×10⁴ to 1×10⁸, 1×10⁵ to 1×10⁷, or 1×10⁶. In some cases, recombinant viruses of the disclosure are infected with subject cells at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷ and 1×10¹⁸ MOI. In some aspects, the amount of pharmaceutical composition comprises about 1×10⁸ to about 1×10¹⁵ recombinant viruses, about 1×10⁸ to about 10¹⁴ recombinant viruses, about 1×10¹⁰ to about 1×10¹³ recombinant viruses or about 1×10¹⁰to about 3×10¹² recombinant viruses. It is recognized that the MOI for administration of rAAV to subject cells may differ substantially from the suitable MOI for production or preparation of rAAV.

The present invention includes pharmaceutical compositions comprising a polynucleotide cassette or gene delivery vector produced by a method described herein and a pharmaceutically-acceptable carrier, diluent or excipient. For example, one embodiment is a pharmaceutical composition comprising a polynucleotide and a pharmaceutically acceptable excipient. In a specific embodiment, the recombinant virus is a recombinant adeno-associated virus (AAV). The subject polynucleotide cassettes or gene delivery vector can be combined with pharmaceutically acceptable carriers, diluents and reagents useful in preparing a formulation that is generally safe, non-toxic and desirable, and includes excipients that are acceptable for primate use. Such excipients may be solid, liquid, semi-solid or in the case of an aerosol composition, gaseous.

Examples of such carriers or diluents may include, but are not limited to, water, saline, Ringer's solutions, dextrose solution and 5% human serum albumin. Supplementary active compounds can also be incorporated into the formulations. Solutions or suspensions used for the formulations may include a sterile diluent such as water for injection, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation, and compounds for the adjustment of tonicity such as sodium chloride or dextrose. The pH can be adjusted with acids or bases, such as hydrochloride acid or sodium hydroxide. In particular embodiments, the pharmaceutical compositions are sterile.

Pharmaceutical compositions suitable for use with the instant compositions and methods further include sterile aqueous solutions or dispersions and sterile powders for the extemporaneous preparation of sterile injectable solutions or dispersions. In some cases, the composition is fluid to the extent that easy syringe ability exists. In certain embodiments, the compositions are stable under the conditions of manufacture and storage. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant such as pluronic acid 0.001% may be used.

For delayed release, the medicament may be included in a pharmaceutical composition which is formulated for slow release, such as in microcapsules formed from biocompatible polymers or in liposomal carrier systems according to methods known in the art.

The term “combination” or the terms “in combination”, “used in combination with” and “combined preparation” as used herein may refer to the combined administration of two or more agents simultaneously, sequentially, or separately. The term “simultaneously” as used herein means that the agents are administered concurrently or at the same time. The term “sequential” as used herein means that the agents are administered one after the other. The term “separate” as used herein means that the agents are administered independently of each other but within a time interval that allows the agents to show a combined, preferably synergistic effect.

Methods of determining expression levels are known in the art. Any method of determining expression level may be used in the methods of the application. Methods of determining expression level include, but are not limited to, immunoassay methods and activity assays.

Methods of determining concentration are known in the art. Any method of determining concentration may be used in the methods of the application. Methods of determining concentration include, but are not limited to, immunoassay methods, activity assays and serial dilutions.

Immunoassay methods for measuring the presence and quantity of a protein in a biological or cell sample are known in the art. See, for example, Hage, D. S. (1999) “Immunoassays” Analytica Chemistry 71(12):294-304; The Immunoassay Handbook, 4^{th} Edition: Theory and Applications of Ligand Binding, ELISA and Related Techniques by David Wild (Ed) Elsevier Science (2013). Immunoassays are generally based on the reaction between a target protein and an antibody or antibody fragment specifically binding to the target protein. Immunoassay may be performed in a liquid or solid phase. Suitable immunoassays include, but are not limited to, sandwich and competition assays, Western blotting, ELISAs, radioimmunoassays, fluoroimmunoassays and the like. The biological sample can be a cell culture medium or supernatant (a sample take from the culture without lysing the cells), cell lysate, whole cells, blood, serum, plasma, aqueous humor, vitreous humor or other body fluid or tissue. It is recognized that a biological sample from a subject may be enriched by separation of whole cells from the sample, particularly when the polypeptide of interest may be secreted from a cell. Separation may be by any convenient separation technique known in the art including, but not limited to, fluorescence activated cell sorting (FACS), magnetic separation, affinity chromatography, “panning” with an affinity reagent, centrifugation and ultracentrifugation.

As used herein, the terms “sequence identity”, “percent identity” and “percent sequence identity” refer to the degree of identity between two or more polynucleotides when aligned using a nucleotide sequence alignment program; or between two or more polypeptide sequences when aligned using an amino acid sequence alignment program.

Similarly, the terms “identical” and percent “identity” when used herein in the context of two or more nucleotide or amino acid sequences refers to two sequences that are the same or have a specified percentage of amino acid residues or nucleotides when compared and aligned for maximum correspondence, for example as measured using a sequence comparison algorithm, e.g., the Smith-Waterman algorithm, etc. or by visual inspection. The percent identity between amino acid sequences may be determined using by, for example, the Needleman and Wunsch (1970, J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6 or 4 and a length weight of 1, 2, 3, 4, 5, or 6. As another example, the percent identity between two nucleotide sequences may be determined using the GAP program in the GCG software package, using a NWSgapdna. CMP matrix and a gap weight of 40, 50, 60, 70, or 80 and length weight of 1, 2, 3, 4, 5 or 6. A particularly preferred set of parameters (and the one that should be used unless otherwise specified) are a Blossum 62 scoring matrix with a gap penalty of 12, a gap extend penalty of 4, and a frameshift gap penalty of 5. The percent identity between two amino acid or nucleotide sequences may also be determined using the algorithm of E. Meyers and W. Miller (1989, Cabios, 4:11-17) which has been incorporated into the ALIGN program (version 2.0) using a PAM120 weight residue table, a gap length penalty of 12, and a gap penalty of 4. Additional search and alignment tools known in the art include, but are not limited to, the NBLAST and XBLAST programs (version 2.0) of Altschul et al (1990) J. Mol. Biol. 215:403-410 and the Gapped BLAST program.

The term “subject”, “patient”, or “individual” refers to a mammal including but not limited to, primates, such as humans and non-human primates, e.g., African green monkeys and rhesus monkeys, mammalian sport animals, mammalian farm animals, mammalian pets and rodents. In some embodiments, the subject is a human.

The terms “treat,” “treating”, “treatment,” “ameliorate” or “ameliorating” and other grammatical equivalents as used herein, refer to alleviating, abating or ameliorating a disease or disorder, or symptoms of a disease or disorder, preventing additional symptoms of the disease or disorder, ameliorating or preventing the underlying causes of symptoms, inhibiting a disease or disorder, e.g., arresting the development of a disease or disorder, relieving a disease or disorder, causing regression of a disease or disorder, or stopping the symptoms of a disease or disorder, and are intended to include prophylaxis and prevention. The terms further include achieving a therapeutic benefit and/or a prophylactic benefit. The term “therapeutic benefit” refers to eradication or amelioration of a disease or disorder being treated. Also, a therapeutic benefit is achieved with the eradication or amelioration of one or more of the physiological symptoms associated with a disease or disorder such that an improvement is observed in the subject, notwithstanding that, in some embodiments, the subject is still afflicted with a disease or disorder. For prophylactic benefit, the pharmaceutical compositions are administered to a subject at risk of developing a disease or disorder, or to a subject reporting one or more of the physiological symptoms of a disease or disorder, even if a diagnosis of the disease or disorder has not been made.

The terms “treat,” “treating”, “treatment,” “ameliorate” or “ameliorating” and other grammatical equivalents as used herein may refer to alleviating, abating or ameliorating dry age-related macular degeneration (dry-AMD) disease or disorder, or symptoms of dry-AMD disease or disorder, preventing additional symptoms of the dry-AMD disease or disorder, ameliorating or preventing the underlying causes of symptoms, inhibiting dry-AMD disease or disorder, e.g., arresting the development of dry-AMD disease or disorder, relieving dry-AMD disease or disorder, causing regression of dry-AMD disease or disorder, or stopping the symptoms of dry-AMD disease or disorder, and may include prophylaxis and prevention of wet-AMD. The terms further include achieving a therapeutic benefit and/or a prophylactic benefit. The term “therapeutic benefit” of dry-AMD refers to eradication or amelioration of dry-AMD disease or disorder being treated. Also, a therapeutic benefit is achieved with the eradication or amelioration of one or more of the physiological symptoms associated with dry-AMD disease or disorder such that an improvement is observed in the subject, notwithstanding that, in some embodiments, the subject is still afflicted with dry-AMD disease or disorder. For prophylactic benefit, the pharmaceutical compositions are administered to a subject at risk of developing dry-AMD disease or disorder, or to a subject reporting one or more of the physiological symptoms of dry-AMD disease or disorder, even if a diagnosis of the disease or disorder has not been made.

Signs and symptoms of dry-AMD include, but are not limited to, endothelial cell proliferation and retinal pigment epithelium (RPE) atrophy.

The terms “administer,” “administering”, “administration,” and the like, as used herein, can refer to the methods that are used to enable delivery of therapeutics or pharmaceutical compositions to the desired site of biological action. These methods include intravitreal or subretinal injection to an eye.

The terms “effective amount”, “therapeutically effective amount” or “pharmaceutically effective amount” as used herein, can refer to a sufficient amount of at least one pharmaceutical composition or compound being administered which will relieve to some extent one or more signs or symptoms of the ocular disease, ocular disorder or ocular condition being treated. An “effective amount”, “therapeutically effective amount” or “pharmaceutically effective amount” of a pharmaceutical composition may be administered to a subject in need thereof as a unit dose (as described in further detail elsewhere herein). The subject may be a human or non-human mammal.

The term “pharmaceutically acceptable” as used herein, can refer to a material, such as a carrier or diluent, which does not abrogate the biological activity or properties of a compound disclosed herein, and is relatively nontoxic (i.e., when the material is administered to an individual it does not cause undesirable biological effects nor does it interact in a deleterious manner with any of the components of the composition in which it is contained).

The term “pharmaceutical composition,” or simply “composition” as used herein, can refer to a biologically active compound, optionally mixed with at least one pharmaceutically acceptable chemical component, such as, though not limited to carriers, stabilizers, diluents, dispersing agents, suspending agents, thickening agents, excipients and the like.

Complement Factor I (also known as Factor I, CFI, and C3b/C4b inactivator) is a protein that, in humans, is encoded by the CFI gene. CFI is a serine protease that circulates in a zymogen-like state typically at a concentration of about 35 μg/mL (Roversi et al (2011) PNAS 108:12839-12844, Nilsson et al (2011) Mol Immunol 48:1611-1620). CFI inactivates C3b by cleaving it into iC3b, C3d and C3d, g and in an analogous way, C4b into C4c and C4d. Thus, CFI activity downregulates complement cascade in all complement pathways (alternative, classical and lectin). CFI requires the presence of one or more cofactor proteins to perform its functions; cofactor proteins include, but are not limited to, C4BP, CFH, CR1 (also known as CR1/CD35) and MCP (CD46); see Degn et al (2011) Am J Hum Genet 88:689-705. Once C3b has been cleaved into iC3b; iC3b does not perpetuate amplification of the complement cascade or activation through the alternative pathway. iC3b promotes a proinflammatory action by activating complement receptor 3 (CR3) on certain cell types. CFI is capable of processing iC3b into C3dg in the presence of the cofactor CR1. C3dg is unable to bind CR3. C3b binding to CR3 is involved with complement activation leading to inflammation; the breakdown of iC3b to C3dg reduces complement-induced inflammation (Lachmann (2009) Adv. Immunol. 104:115-149). CFI is capable of processing iC3b into an inactive degradation product.

Ocular diseases and disorders are known in the art. Ocular diseases and disorders include, but are not limited to, achromatopsia, glaucoma, retinitis pigmentosa, macular degeneration, retinoschisis, Leber's Congenital Amaurosis, diabetic retinopathy, color blindness, diabetic macular edema, choroidal neovascularization, proliferative diabetic retinopathy, retinal vein occlusion, central retinal vein occlusion, branched retinal vein occlusion, diabetic macular edema, diabetic retinal ischemia, ischemic retinopathy, diabetic retinal edemaMacular degeneration may include, but is not limited to, dry macular degeneration, wet macular degeneration, age-related macular degeneration and acute macular degeneration.

Age-related macular degeneration (AMD) is a degenerative ocular disease affecting the macula, a light sensitive, small area in the center of the retina that is responsible for reading and high acuity. Conditions affecting the macula reduce central vision while leaving peripheral vision intact. In severe cases, the disease can lead to central blindness. AMD is a notable cause of vision loss in the US population among persons 65 years and older, and the estimated prevalence of any AMD among persons over 40 years of age is approximately 6.5% (Klein et al., (2011) Arch Ophthalmol, 129(1): 75-80).

There are two forms of age-related macular degeneration, dry (atrophic) and wet macular degeneration. Dry-AMD is more common than wet-AMD, but the dry can progress to wet-AMD. Dry-AMD is characterized by thinning of the tissues of the macula as cells disappear; dry-AMD may affect both eyes. Dry AMD is typically characterized by progressive apoptosis of the cells in the retinal pigment epithelium (RPE) layer, overlying photoreceptor cells, and frequently also the underlying cells in the choroidal capillary layer. Confluent areas of RPE cell death accompanied by overlying photoreceptor atrophy are referred to a geographic atrophy (GA). As dry-AMD progresses and GA increases, central vision slowly worsens and the ability to see fine detail is gradually lost. Dry AMD tends to progress more slowly than wet AMD.

In some embodiments, the ocular neovascular disease is recurrent and/or persistent wAMD. In some embodiments, the ocular neovascular disease is active subfoveal CNV secondary to AMD. In some embodiments, the active subfoveal CNV secondary to AMD occupies ≥50% of the total lesion size. In some embodiments, the active subfoveal CNV secondary to AMD occupies ≥50% of the total lesion size with evidence of leakage on fluorescein angiogram (FA), fluid on spectral domain optical coherence tomography (SD-OCT), and/or subretinal hemorrhage on color fundus photography. In some embodiments, the active subfoveal CNV secondary to AMD occupies ≥50% of the total lesion size with evidence of leakage on fluorescein angiogram (FA), fluid on spectral domain optical coherence tomography (SD-OCT), and/or subretinal hemorrhage on color fundus photography, and the entire dimension of the lesion does not exceed 12 macular photocoagulation study disc areas. In some embodiments, the one eye and/or the contralateral eye of the individual exhibited best corrected visual acuity (BCVA) based on an ETDRS letters assessment of 78-25 (e.g., less than any of about 78, about 75, about 70, about 65, about 60, about 55, about 50, about 45, about 40, about 35, about 30, or about 25) prior to administration of the unit dose of rAAV particles of the present disclosure. In some embodiments, the one eye and/or the contralateral eye of the individual exhibited best corrected visual acuity (BCVA) based on an ETDRS letters assessment of more than any of about 5, about 10, about 15, about 20, about 25, about 30, about 35, about 40, about 45, about 50, about 55, about 60, about 65, about 70, about 75, about 80, about 85, about 90, about 95, or about 100 prior to administration of the unit dose of rAAV particles of the present disclosure.

In some embodiments, the individual had polypoidal choroidal vasculopathy (PCV) in the one eye and/or the contralateral eye prior to administration of the unit dose of rAAV particles.

Achromotopsia is a rare autosomal recessive disease that results in retinal degeneration affecting all three types of cone photoreceptor cells that results in reduced visual acuity, photophobia, hemeralopia, and severe loss of color discrimination.

Mutation in the CNGB3 genes accounts for greater than 90% of patient and results in complete Achromatopsia, meaning that they have significant impairment in color discrimination and central visual acuity.

In some embodiments, a unit dose of rAAV particles is administered in combination with steroid treatment. In some embodiments, the steroid treatment is a corticosteroid treatment. In some embodiments, the steroid treatment is a systemic steroid treatment. In some embodiments, the steroid treatment is an oral steroid treatment. In some embodiments, the steroid treatment is a prednisone treatment. In some embodiments, the steroid treatment is an ophthalmic steroid treatment. In some embodiments, the ophthalmic steroid treatment is a topical steroid treatment (e.g., a drop), a periocular steroid treatment (e.g., subtenons, subconjunctival), an intravitreal steroid treatment, or a superchoroidal steroid treatment. In some embodiments, the ophthalmic steroid treatment is a glucocorticoid including, but not limited to, an anti-inflammatory glucocorticoid. In some embodiments, the topical steroid treatment is a glucocorticoid including but not limited to, an anti-inflammatory glucocorticoid. In some embodiments, the topical steroid treatment is a difluprednate treatment, a medrysone treatment, a loteprednol treatment, a prednisolone treatment, a fluocinolone treatment, a triamcinolone treatment, a rimexolone treatment, a dexamethasone treatment, a fluorometholone treatment, a fluocinolone treatment, a rimexolone treatment, or a prednisone treatment. Anti-inflammatory glucocorticoids may include, but are not limited to, difluprednate, dexamethasone, prednisolone, triamcinolone, fluorometholone, rimexolone, fluocinolone, loteprednol and bioequivalents thereof. In some embodiments, the topical steroid treatment is a difluprednate treatment. By “dexamethasone” is intended dexamethasone, dexamethasone biosimilars, dexamethasone bioequivalents, and pharmaceutical compositions comprising dexamethasone, a dexamethasone biosimilar or a dexamethasone bioequivalent. Pharmaceutical compositions comprising dexamethasone include, but are not limited to, Ozurdex™, Maxidex™, Decadron™, Dexamethasone Intensol™, Ocu-Dex™, Dexycu™, Dextenza™ and Zodex™ Ozurdex™ is a pharmaceutical composition comprising dexamethasone. By “difluprednate” is intended difluprednate, difluprednate biosimilars, difluprednate bioequivalents, and pharmaceutical compositions comprising difluprednate, a difluprednate biosimilar or a difluprednate bioequivalent. Pharmaceutical compositions comprising difluprednate include, but are not limited to, Durezol™ and difluprednate emulsions. By “triamcinolone” is intended triamcinolone, triamcinolone biosimilars, triamcinolone bioequivalents, and pharmaceutical compositions comprising triamcinolone, a triamcinolone biosimilar or a triamcinolone bioequivalent. Pharmaceutical compositions comprising triamcinolone include, but are not limited to, Triesence™, Xipere™, and Trivaris™. In some embodiments, the steroid treatment is administered before, during, and/or after administration of the unit dose of rAAV particles. In some embodiments, the steroid treatment is administered before administration of the unit dose of rAAV particles. In some embodiments, the steroid treatment is administered during administration of the unit dose of rAAV particles. In some embodiments, the steroid treatment is administered after administration of the unit dose of rAAV particles. In some embodiments, the steroid treatment is administered before and during administration of the unit dose of rAAV particles. In some embodiments, the steroid treatment is administered before and after administration of the unit dose of rAAV particles. In some embodiments, the steroid treatment is administered during, and after administration of the unit dose of rAAV particles. In some embodiments, the steroid treatment is administered before, during, and after administration of the unit dose of rAAV particles.

In some embodiments, the steroid treatment is an ophthalmic steroid treatment (e.g., difluprednate). In some embodiments, the ophthalmic steroid treatment (e.g., difluprednate) is a daily steroid treatment for up to about 4 weeks, about 6 weeks, or about 8 weeks from administering the unit dose of rAAV particles. In some embodiments, the ophthalmic steroid treatment comprises about four administrations of ophthalmic steroid on about week 1, about three administrations of ophthalmic steroid on about week 2, about two administrations of ophthalmic steroid on about week 3, and about one administration of ophthalmic steroid on about week 4; timing starting with and following administration of the unit dose of rAAV particles. In some embodiments, the ophthalmic steroid is about 0.005% to about 0.5% difluprednate. In some embodiments, the ophthalmic steroid is any of about 0.005%, about 0.006%, about 0.007%, about 0.008%, about 0.009%, about 0.01%, about 0.02%, about 0.03%, about 0.4%, about 0.05%, about 0.06%, about 0.07%, about 0.08%, about 0.09%, or about 0.1% difluprednate. In some embodiments, the ophthalmic steroid is difluprednate 0.05%. In some embodiments, a dose of difluprednate 0.05% is one drop of ophthalmic solution. In some embodiments, one drop is about 50 μl (e.g., about 25 μl to about 50 μl, about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

In some embodiments, the steroid treatment is an ophthalmic steroid treatment (e.g., difluprednate). In some embodiments, the ophthalmic steroid treatment (e.g., difluprednate) is a daily topical steroid treatment for up to about 4 weeks, about 6 weeks, or about 8 weeks from administering the unit dose of rAAV particles. In some embodiments, the topical steroid treatment comprises about four administrations of topical steroid on about week 1, about three administrations of topical steroid on about week 2, about two administrations of topical steroid on about week 3, and about one administration of topical steroid on about week 4; timing starting with and following administration of the unit dose of rAAV particles. In some embodiments, the topical steroid treatment comprises about four administrations of topical steroid (i.e., QID) per day for about 3 weeks after administration of the unit dose of rAAV particles, followed by about 3 administrations of topical steroid per day (i.e., TID) for about 1 week, followed by about 2 administrations of topical steroid per day (i.e., BID) for about 1 week, and followed by about 1 administration of topical steroid per day (i.e., QD) for about 1 week. In some embodiments, the topical steroid comprises difluprednate 0.05% at a dose of about lug to about 3μg. In some embodiments, the topical steroid comprises difluprednate 0.05% at a dose of about 2.5μg. In some embodiments, the topical steroid is about 0.005% to about 0.5% difluprednate. In some embodiments, the topical steroid is any of about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1% difluprednate. In some embodiments, the topical steroid is difluprednate 0.05%. In some embodiments, a dose of difluprednate 0.05% is one drop of ophthalmic solution. In some embodiments, one drop is about 50 μl (e.g., about 25 μlto about 50 μl, about 50 μlto about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

The methods, systems and kits described herein may employ, unless otherwise indicated, conventional techniques and descriptions of molecular biology (including recombinant techniques), cell biology, biochemistry, immunochemistry and virology techniques which are within the skill of those who practice the art. Such conventional techniques include methods for cloning and propagating recombinant virus, formulation of a pharmaceutical composition and biochemical purification and immunochemistry. Specific illustrations of suitable techniques can be had by reference to the examples herein. However, equivalent conventional procedures may also be used. Such conventional techniques and descriptions can be found in standard laboratory manuals such as Weiner et al., Eds, Genetic Variation: a Laboratory Manual (2007); Dieffenbach, Dveksler (Eds.), PCR Primer: a Laboratory Manual (2003); Sambrook and Russell, Condensed Protocols from Molecular Cloning: a Laboratory Manual (2006); Miller & Calos eds. (1987) “Gene Transfer Vectors for Mammalian Cells” and Current Protocols in Immunology (1999-2022) John Wiley & Sons, all of which are hereby incorporated by reference in their entirety for all purposes.

It will be understood that the reference to the below examples is for illustration purposes only and do not limit the scope of the claims. Any of a number of alternative compositions and methods are applicable and suitable for using in practicing the claimed methods and compositions. It is also understood that an evaluation of the expression constructs and methods of the application may be carried out using procedures standard in the art.

### EXAMPLES

### Example 1

### Cell Culture

Sf9 rhabdovirus-negative (Sf-RVN®) insect cells (GlycoBac) were substituted for Sf9 insect cells to increase the titer of rAAV production. Sf-RVN cells were maintained with constant rotation at 125 rpm at 28° C. in polycarbonate Erlenmeyer flasks (Corning) using serum-free Sf-900IITM SFM (GibcoTM) or ESF AF (Expression Systems) media in Multitron shaker (Infors HT). Cells were verified to be in the log phase (3.0-4.0E6 cells/mL) with greater than 90% viability using Vi-Cell BLU (Beckman Coulter).

### Example 2

### Recombinant Baculovirus Construction

Recombinant baculoviruses were constructed using the Bac-to-Bac Baculovirus Expression System (Invitrogen). This method is based on the site-specific transposition of donor plasmid (pFastBac™) sequences into a baculovirus shuttle vector (bacmid) propagated in DH10Bac™ E. coli cells. pFastBac™ vectors containing the gene(s) of interest or rep-cap, ampicillin resistance gene, and Autographa californica multiple nuclear polyhedrosis virus (AcMNPV) polyhedrin (polh) and P10 promoters for high-level expression in insect cells were ordered from Integrated DNA Technologies. The pFastBac™ expression cassette is flanked by the left and right arms of Tn7, and also contains a gentamicin resistance gene and an SV40 polyadenylation signal to form a mini Tn7. In our modified system, the gentamicin resistance gene and the GC-rich regions close to the Tn7 sites were removed to avoid unnecessary transfer of antibiotic resistance genes into patients and also prevent the occurrence of inflammation.

The pFastBac™ vector was transformed into the DH10Bac™ E. coli strain, which contains a baculovirus shuttle vector (bacmid) with a mini-attTn7 target site and a helper plasmid, using the heat shock method. 1 μlof 1mg/ml pFastBac™ vector was added into 20 μlDH10Bac™ cells (Invitrogen) thawed on ice. Cells were incubated on ice for 30 minutes, then were transferred into a water bath (Poly Science) at 40° C. for 25 seconds. Next, cells were returned to the ice for 2 minutes followed by the addition of 1000 μl SOC media (Thermo Scientific). The DH10Bac™ cells containing the plasmid were grown for 6 hours at 37° C. with 220 rpm in an Innova43 shaker (New Brunswick). Transposition of the desired sequence occurs between the mini-Tn7 element on the pFastBac™ vector and the mini-attTn7 target site on the bacmid to generate a recombinant bacmid (rbacmid) in the presence of transposition proteins supplied by the helper plasmid. After 6 hours, cells were spread on an agar plate (Teknova) and incubated at 37° C. for 48 hrs. The agar plate contains 50 μg/ml kanamycin and 10 μg/ml tetracycline as the bacmid and pHelper confers resistance to kanamycin and tetracycline respectively. The agar plate also contains 200 μg/ml Isopropyl β-D-1-thiogalactopyranoside (IPTG) and 20 μg/ml Blue-gal which is a chromogenic substrate. This helps with the screening of the clones containing bacmid using blue vs white colonies. Upon the formation of the recombinant bacmid, lacZ deletion on the bacmid completes which results in no production of ß-galactosidase from the reporter cassette. Otherwise, β-galactosidase produced by lacZ would hydrolyze the Blue-gal to form 5-bromo-4-chloro-indoxyl, which produces an insoluble blue pigment called 5,5′-dibromo-4,4′-dichloro-indigo. The colonies formed by non-recombinant cells, therefore appear blue while the recombinant ones appear white.

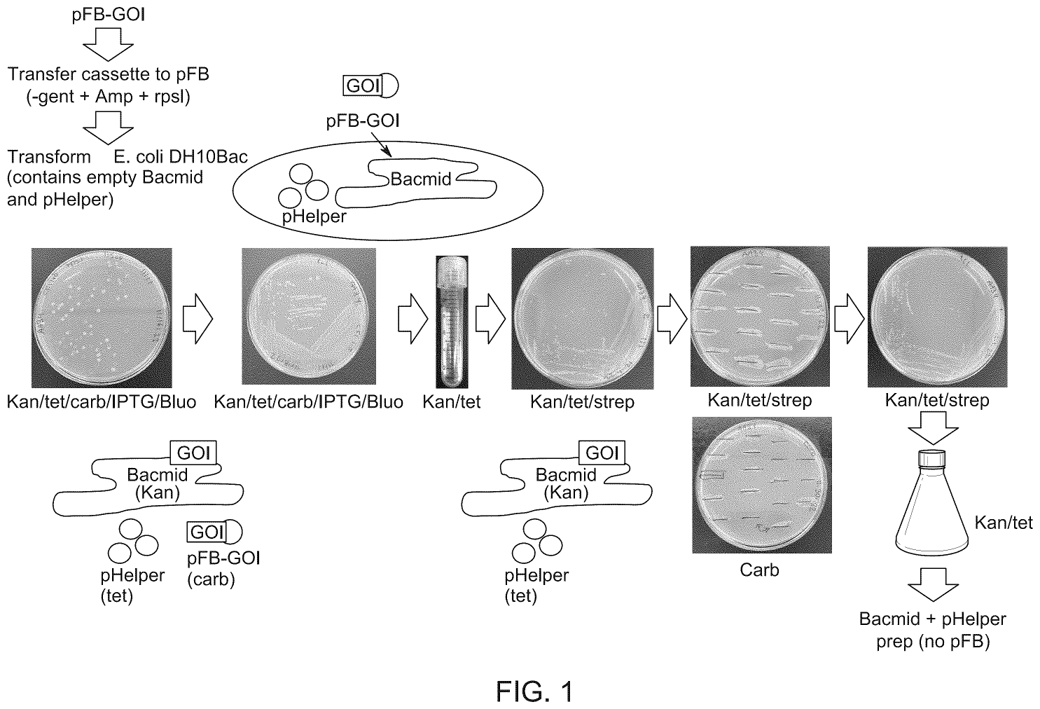

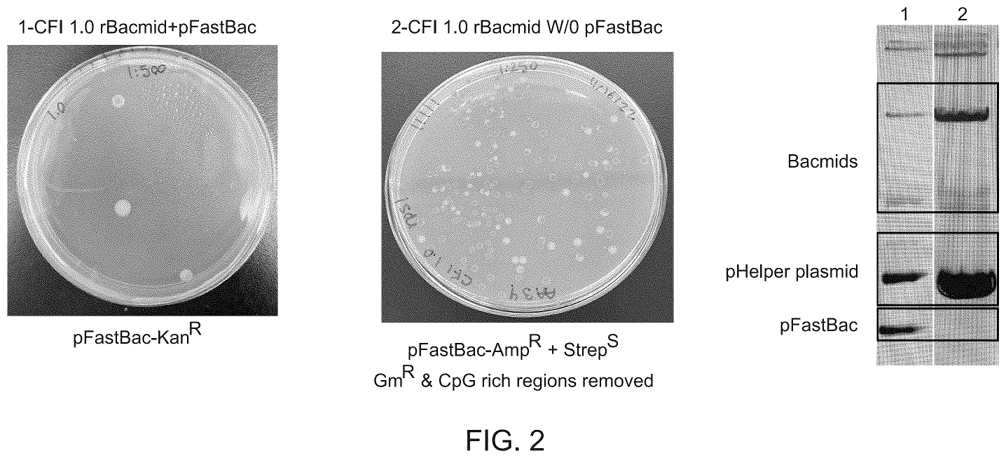

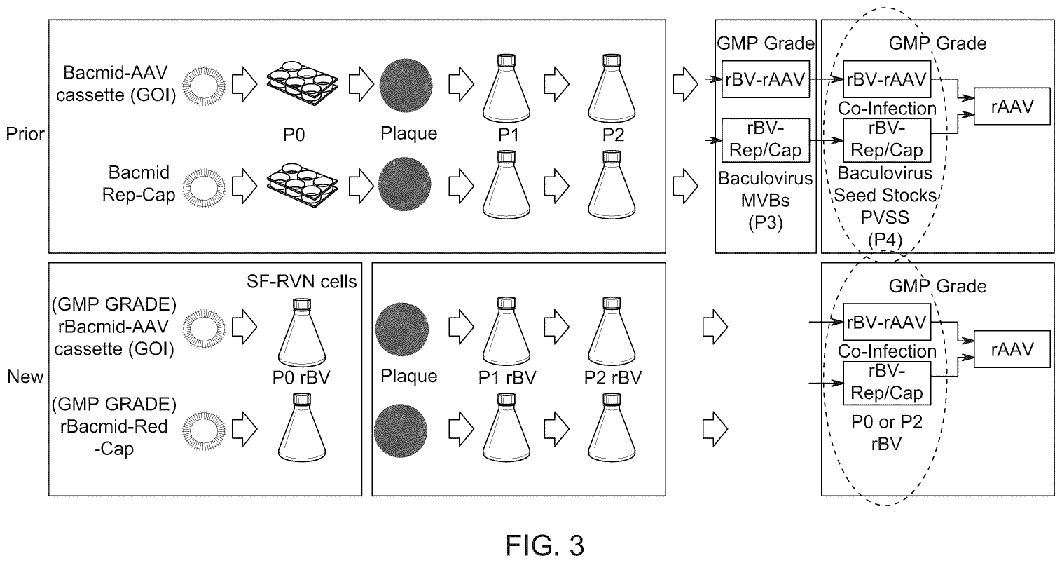

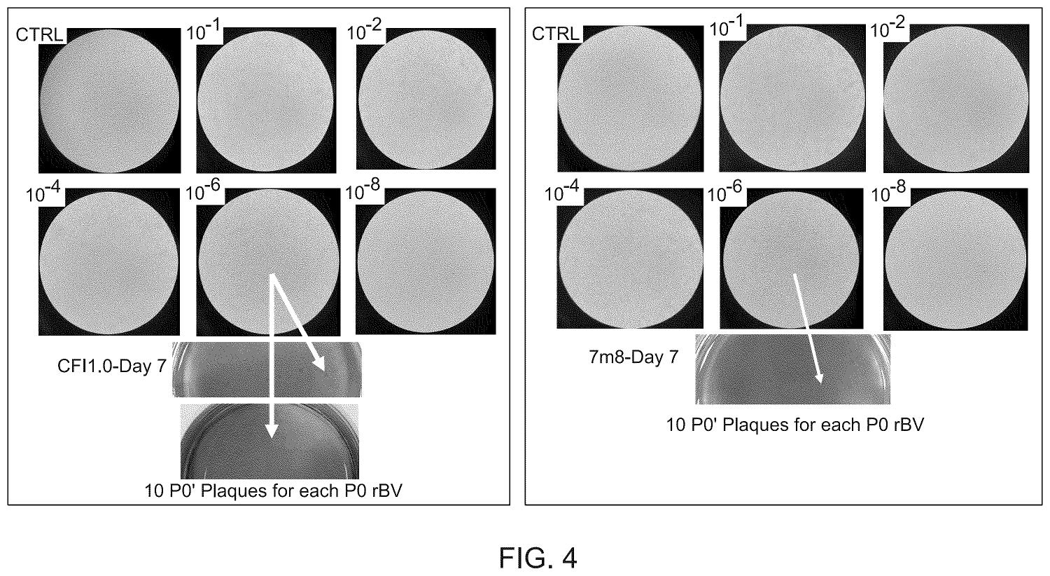

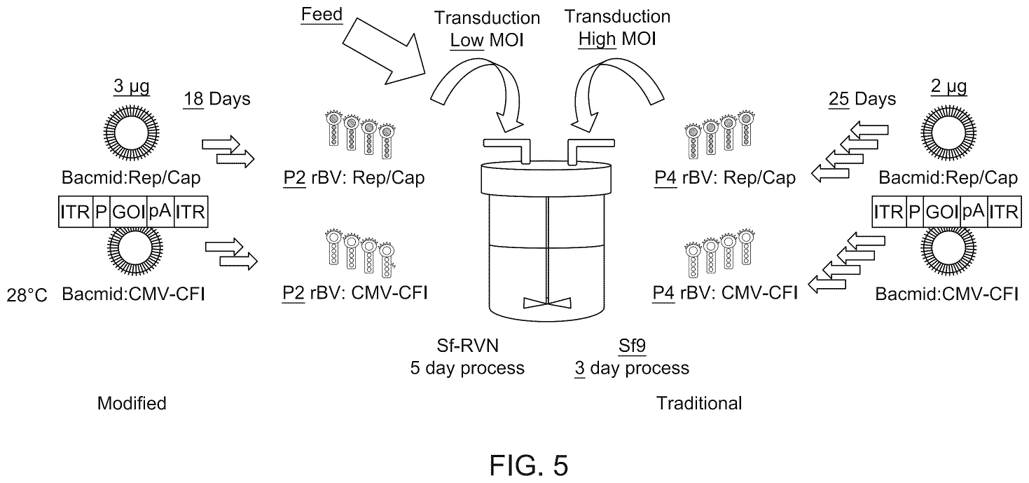

A single white colony was restreaked on a new agar plate with the same ingredients and let grow for 48 hrs to confirm the color. In the original Bac-to-Bac system, a single colony from the 2nd agar plate is used to purify the bacmid. However, we have modified the system in a way to remove the pFastBac™ from the colonies. To do so, a streptomycin sensitive gene (rpsl) which works as a counter-selective marker was added to the pFastBac™. After white colonies were confirmed on the 2nd agar plate, single colonies were propagated on the 3rd agar plate which contained 50 μg/ml kanamycin, 10 μg/ml tetracycline, and 100 μg/ml streptomycin. Each of the colonies on the 3rd plate was grown on the 4th agar plate with 100 μg/ml carbenicillin. One of the colonies that were not able to grow on the carbenicillin plate was chosen for the next step. The colony was spread on the 5th plate with the 3 antibiotics. Next, a single colony from the last plate was grown overnight in 170 mL 2XYT Broth with Animal-free soytone media (Teknova) with Kanamycin and tetracycline at 37° C. with 220 rpm in Innova 43 shaker. The bacmid from the culture was purified using PureLink™M HiPure Plasmid Filter Maxiprep Kit (Invitrogen) following the manufacturer's protocol. An overview of a process with representative steps is shown in FIG. 1. The presence of rbacmid and absence of donor plasmid was confirmed using 0.5% agarose gel and ddPCR. See FIG. 2.

### Example 3

### PO Passage of Recombinant Baculovirus (rBV)

Purified rBacmid DNA from the bacterial colony was used to transfect Sf-RVN cells to generate recombinant baculovirus. rBacmid GFP/Fluc rBacmid was designed as gene of interest which GFP/Fluc reporter gene flanked by ITRs. The 7m8 rBacmid was designed to include polh and P10 promoter-driven modified-AAV2 Rep/Cap genes.

Sf-RVN Cells were verified to be in the log phase (3.0-4.0E6 cells/mL) with greater than 90% viability using Vi-Cell BLU. Cells were centrifuged at 230 G for 10 minutes and resuspended in fresh SF-900 II SFM at a density of 2E6/mL. 9 μg rbacmid was added into 1 mL 1× PBS (pH 7.4, no Cal, no Mag) (GibcoTM), and 45 μL Cellfectin™ II Reagent (Gibco™) reagent was added into another 1 mL 1× PBS. Mixtures were incubated for 5 mins and then the rbacmid was added to the Cellfectin and incubated for 30 minutes at room temperature. Cellfectin reagent needs to be at room temperature and well mixed before use. After 30 mins, the DNA-oil mixture was added to 30 mL of the cells drop-wise. At day 4 post-transfection, cells were checked with Vi-Cell BLU, then centrifuged for 10 minutes at 230 G using a Sorvall Legend XFR centrifuge (Thermo Scientific). The supernatant was collected in amber canonical centrifuge tubes (Avantor) as the PO stock of rBVs. 5% sucrose was added to the rB Vs for long-term storage at −80° C. The titer of the rBVs was checked using ddPCR.

### Example 4

### Plaque Assay Purification

After Sf-RVN cells were verified to be in the log phase (3.0-4.0E6 cells/mL) with greater than 90% viability they were resuspended in Sf-900II SFM at 0.5E6/ml density. 2 mL of the diluted cells were added to each well of a 6 well-plate and incubated at room temperature for 30 minutes. 4% agarose gel (Gibco) was transferred from 4° C. into the 70° C. water bath. A 100 mL empty bottle and Sf-900™ 1.3X (Gibco) were placed into the 40° C. water bath. Next, serial dilution of PO rBVs at E10 vg/ml was performed using SF900II SFM from 10⁻¹ to 10⁻⁷. One skilled in the art recognizes that 1E# notation format is equivalent to the 1×10# notation format.

After cells are attached to the 6-well plate they were checked with an inverted microscope (Zeiss Invertroskop) for 50-70% cell confluency. Next, 1000 μL Sf 900II SFM was added to the first well as the CTRL, and 1000 L of dilutions 10⁻³ to 10⁻⁷ was added to the corresponding wells. The 6-well plate was incubated at 28° C. for 1 hour.

After the agarose melted, 30 mL of the SF-900 (1.3×) medium and 10 mL of the 4% agarose gel were added to the empty bottle and mix gently to avoid the creation of bubbles. This made the 1% agarose overlay which was returned to the 40° C. water bath until use.

At the end of 1 hr incubation, the virus was aspirated and replaced with 2 mL of 1% agarose overlay. The agarose overlay was allowed to solidify in BSC for 20 minutes. Next, 1 mL of SF900 II SFM was added on top of the agarose overlay and the plate was incubated at 28° C. for 7-10 days. At Days 7-10, depending on plaque formation and growth, the medium was removed from the agarose overlay and individual plaques were harvested using a 2mL pipet by inserting the tip into the agarose overlay with gradual suction so that the agarose plugs and some liquids are sucked into the tip. The agarose plugs were transferred into sterile tubes containing 1 mL SF900II SFM, pipetting up and down several times to rinse the agarose and liquid into the sterile tubes. The tubes were kept at 4° C. overnight to produce PO rBVs or BEVs.

### Example 5

### P1 Passage of rBVs

One day before infection, Sf-RVN cells were checked to be in the log phase (3.0-4.0E6 cells/mL) with greater than 90% viability using Vi-Cell BLU. 30 mL cells were cultured in 125 mL flasks at 1.2E6/ml density using SF900II SFM for each P0′ rBV. On the Day of Infection, cells were verified again using Vi-Cell BLU and 500 uL ofP0O′ rBVs was added to each flask. At day 4 post-infection, cells were harvested as described in Example 3. ddPCR was performed on P1 rBVs to determine the most stable clones.

### Example 6

### P2 Passage rB Vs

P2 passage were made as described in Example 5 using the most stable clones of P1 rBVs for Gene of Interest (GOI) and Rep-Cap. However, to improve the consistency of the protocol, instead of 1 to 1000 dilution of P1 rBVs that was used previously, MOI 0.1 was used to infect the Sf-RVN cells. At day 3 post-infection, P2 rBVs were harvested and ddPCR was done to determine the vg titer of the viruses.

### Example 7

### Media and MOI Optimization

To optimize the rAAV generation, SF900II SFM, SF900III SFM (Gibco), ESF-AF, and EX-Cell CD (SAFC) media were used to find the highest rAAV titer. ESF-AF medium resulted in the highest rAAV titer in the range of E12 vg/ml (E8 IFU/ml). Therefore, the ESF-AF medium was chosen for rAAV production.

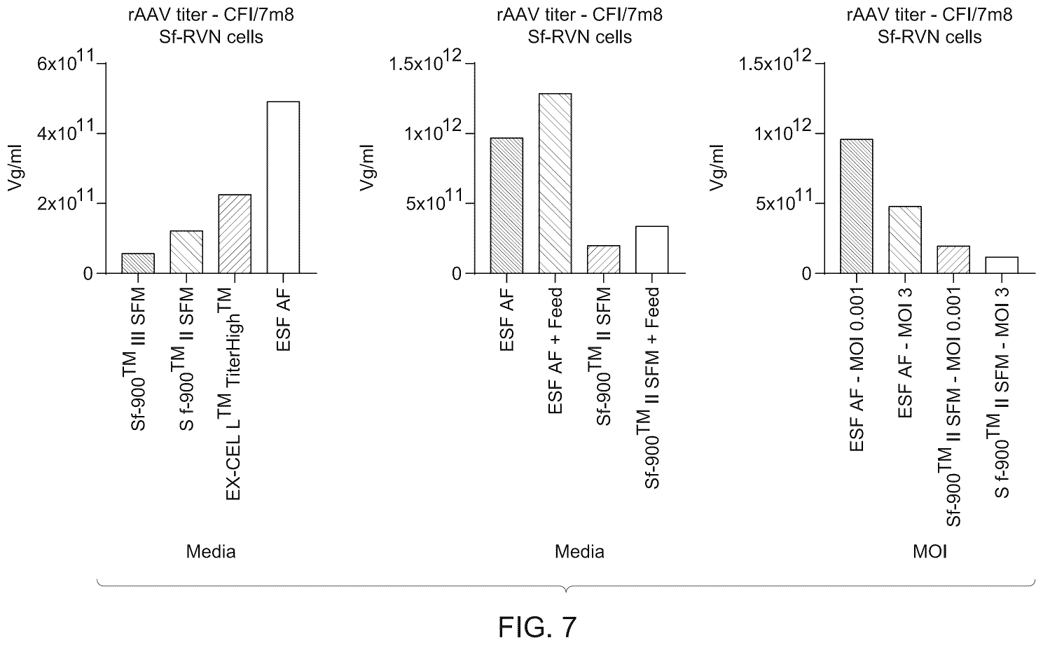

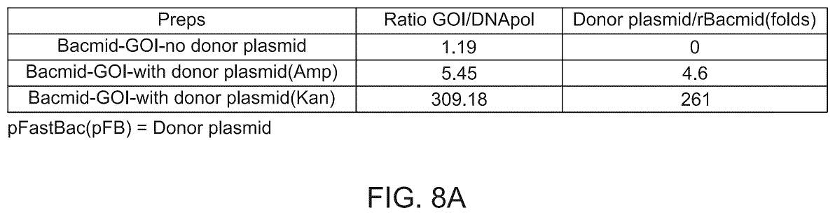

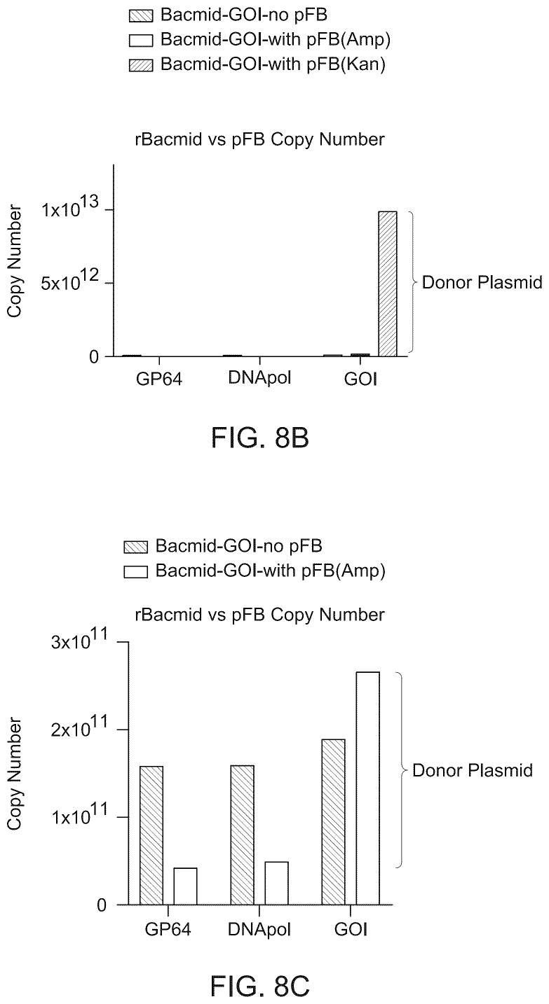

Insect cells were co-infected with 7m8 rBV and CFI rBV at a MOI of either 3 or 0.001 in two media types (SF900II SFM or ESF-AF). rAAV were harvested and the rAAV titers were determined. The rAAV titer obtained from co-infection at a MOI of 0.001 was significantly higher than that obtained from co-infection at a MOI of 3. Results from one such experiment are presented in FIG. 7.

### Example 8

### rAAV Production

One day before infection, Sf-RVN Cells in ESF-AF media were verified to be in the log phase (3.0-4.0E6 cells/mL) with greater than 90% viability and were prepared at 1.2E6/ml density in 30 mL cultures for each rAAV production. The next day, P2 rBVs carrying the GOI and P2 rBVs carrying the Rep/Cap were added to the cells at MOI 0.001. Previous methods used MOI 5. After cells were incubated in the shakers at 28° C. for 4 hours, 225 μL immediate Advantage™ 66765 40× insect feed (SAFC) and 4.5 μL fatty acid (SAFC) were added to each culture. At day 5 post-infection, cells were checked using Vi-Cell BLU. For harvesting, 10× AAV-MAX lysis buffer (Gibco), to a final 1× concentration in culture, 1M MgCl₂ to a final 2-4 mM concentration in culture and 90 U/mL Benzonase were added to each 30 mL culture. Next, cultures were incubated in the shaker at 37° C. for 2 hours. After cells were lysed, they were pelleted at 4000 rpm for 30 mins in a Sorvall Legend XFR centrifuge. The supernatant was filtered using a 0.2 μm a PES membrane (Thermo Scientific) and stored at −80° C. for later purification. The titer of the rAAV viruses was determined using ddPCR.

In some instances, insect feed was not added to the cultures. rAAV titers obtained from methods with and without feed addition were determined. The addition of feed increased the rAAV titers obtained from both insect cells grown in both ESF AF media and Sf-900II™ SFM media. Results from one such experiment are shown in FIG. 7.

### Example 9

### Evaluation of rBV Stability During Different Passages and Effect of Gene of Interest Stability on rAAV Titer

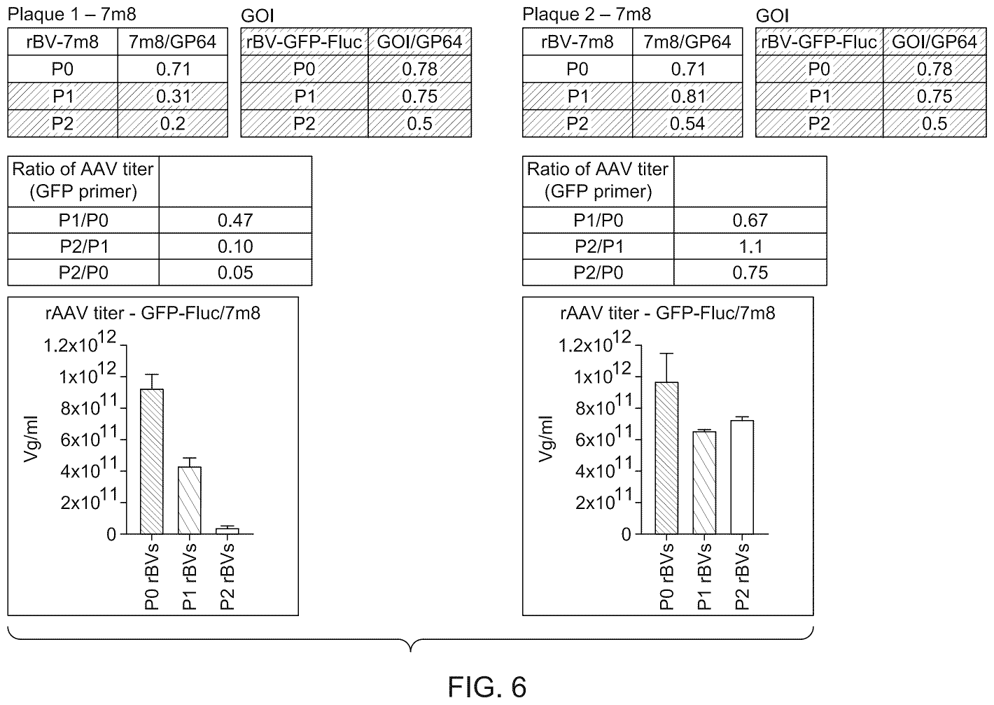

After generating PO rBV, the plaque purification assay was performed and 10 different plaques for GOI (GFP-Fluc) and 7m8 were selected. The gene of interest/vector backbone ratio in 10 different plaques was evaluated. Results from one such experiment are presented in FIG. 6.

For the rBV-GOI (GFP-Fluc), 78% of the rBV retain the gene of interest at passage 0 (P0), 75% of the rBV retain the gene of interest after passage 1 (P1) and 50% of the rBV retain the gene of interest after passage 2 (P2).

For the rBV-7m8, two different plaques were selected for further evaluation. In 7m8 plaque 1, 71% of the rBV retain the rep/cap gene of interest at P0, 31% of the rBV retain the rep/cap gene of interest at P1 and 20% of the rBV retain the rep/cap gene of interest at P2.

In 7m8 plaque 2, 71% of the rBV retain the rep/cap gene of interest at P0, 81% of the rBV retain the rep/cap gene of interest at P1 and 54% of the rBV retain the rep/cap gene of interest at P2.

rAAV were produced with P0, P1 and P2 rBVs (BEVs) from two different 7m8 plaques (plaque 1 and 2) with with the same GOI plaque (rBV-GOI: GFP-Fluc). rAAV titer with Plaque 1-7m8 drops by 20-fold however with plaque 2-7m8, rAAV titer only drops around 25%.

### Example 9

### Evaluation of pHelper Effect on Stable Gene of Interest Formation

Donor plasmids comprising a gene of interest, bacmids and pHelper plasmids a are transformed into bacterial cells. The bacterial cells are transformed with multiple amounts of pHelper plasmid. The bacterial cells are grown under selection for all three plasmids and in conditions that allow identification of recombinant bacmids. In some experiments, bacteria are transformed with a standard amount of pHelper and grown in media with varying amounts of an antibiotic to which the pHelper provides resistance. Bacteria from bacterial colonies comprising recombinant bacmids are grown under conditions that counter select for the donor plasmid. Bacmids are harvested from bacterial colonies comprising recombinant bacmids. Recombinant bacmids are evaluated by next generation sequencing (NGS) of long read sequences. The recombinant bacmids comprising concatamers are identified. The copy number of the gene of interest is evaluated.

Insect cells are infected with recombinant bacmids. PO rBV are obtained. NGS is used to evaluate the copy number of the gene of interest. The population heterogenicity is evaluated. P0 rBVs are infected in insect cells and Pl rBV are harvested. NGS is used to evaluate the copy number of the gene of interest. The population heterogenicity is evaluated in the P1 rBV population.

1. A system for production of recombinant bacmids with reduced residual DNA, said system comprising:
i. a donor plasmid comprising a first antibiotic resistance gene, a counter selective marker and a transfer cassette, wherein the transfer cassette comprises a left transposon arm, a gene of interest and a right transposon arm;
ii. a helper plasmid comprising a second antibiotic resistance gene;
iii. a bacmid comprising third antibiotic resistance gene and a transposon insertion site located within a reporter cassette.

2.-5. (canceled)

6. The system of claim 1, wherein the counter selective marker is an antibiotic sensitive gene.

7. The system of claim 6, wherein the antibiotic sensitive gene is selected from the group comprising rpsl, URA3 and thymidine kinase.

8. The system of claim 6, wherein the antibiotic sensitive gene is rpsl.

9. The system of claim 1, wherein the donor plasmid does not comprise an active gentamycin resistance gene.

10. The system of claim 1, wherein the transfer cassette is optimized to reduce GC rich regions in the transfer cassette.

11. The system of claim 1, wherein the donor plasmid comprises neither an active gentamycin resistance gene nor a GC rich region in the transfer cassette.

12.-17. (canceled)

18. The system of claim 1, wherein the system is selected from the group comprising a modified Bac-to-Bac system, a modified baculovirus infectious cells (BIC) system and a modified Bac-plus system.

19-29. (canceled)

30. An efficient-method of producing a recombinant baculovirus seed stock comprising a gene of interest, wherein at least 50% of the recombinant baculovirus (rBV) in the recombinant baculovirus seed stock comprise a gene of interest, said method comprising the steps of:
(a) introducing a system for production of recombinant bacmids comprising a gene of interest into bacterial cells, wherein the system comprises
a) a donor plasmid comprising a first antibiotic resistance gene, a counter selective marker and a transfer cassette, and wherein the transfer cassette comprises a left transposon arm, a gene of interest and a right transposon arm;
b) a helper plasmid comprising a second antibiotic resistance gene;
c) a bacmid comprising third antibiotic resistance gene and a transposon insertion site located within a reporter cassette;
(b) growing the transformed bacteria in the presence of three antibiotics to which the first, second and third antibiotic resistance genes confer resistance;
(c) selecting at least one bacterial colony comprising a recombinant bacmid;
(d) incubating bacteria from at least one bacterial colony comprising a recombinant bacmid selected in step (c) with a compound that allows counter selection against the donor plasmid;
(e) collecting recombinant bacmids comprising a gene of interest from a bacterial colony after counter selection;
(f) transforming insect cells with the recombinant bacmids comprising a gene of interest collected in step (e);
(g) incubating the transformed insect cells and harvesting rBV comprising the gene of interest from the insect cells;
(h) performing a first passage, wherein a passage comprises the steps of infecting insect cells with rBV comprising the gene of interest and incubating the infected insect cells;
(i) harvesting rBV comprising the gene of interest from at least two plaques of insect cells and identifying a plaque comprising rBV comprising a gene of interest and wherein the rBV exhibit a low rate of loss of the gene of interest; and
(j) performing a second passage (P1 rBV), wherein the second passage comprises infecting insect cells with rBV from the plaque identified in step (i), incubating the infected insect cells and harvesting a recombinant baculovirus seed stock comprising rBV comprising the gene of interest from the insect cells.

31. The method of claim 30, wherein after the first passage at least 80% of the rBV comprise the gene of interest.

32. (canceled)

33. The method of claim 30, wherein said method produces a recombinant baculovirus seed stock after two passages through insect cells, wherein the recombinant baculovirus seed stock is at least 2×10¹⁰ vector genomes/ml (vg/ml).

34. The method of claim 30, wherein a bacterial colony comprising a recombinant bacmid is identified by an alteration in reporter activity.

35.-38. (canceled)

40. The method of claim 30, wherein the first antibiotic resistance gene, the second antibiotic resistance gene and the third antibiotic resistance gene are selected from the group comprising kanamycin resistance genes, ampicillin resistance genes, tetracycline resistance genes, penicillin resistance genes, streptomycin resistance genes, erythromycin resistance genes and penicillin/streptomycin resistance genes.

41. The method of claim 30, wherein donor plasmid comprises an ampicillin resistance gene and rpsl, the helper plasmid comprises a tetracycline resistance gene and the bacmid plasmid comprises a kanamycin resistance gene.

42. The method of claim 30, wherein the compound that allows counter selection against the donor plasmid is an antibiotic.

43.-45. (canceled)

46. The method of claim 30, wherein the insect cells are selected from the group comprising Sf-RVN cells, Sf9 cells and Hi5 cells.

47. (canceled)

48. The method of claim 30, wherein the insect cells are transformed with between about 1 μg and 20 μg bacmids.

49-54. (canceled)

55. A method of producing a recombinant baculovirus seed stock comprising a stable gene of interest, wherein at least 50% of the recombinant baculovirus (rBV) in the recombinant baculovirus seed stock comprise a stable gene of interest, the method comprising the steps of:
(a) introducing a system for production of recombinant bacmids comprising a gene of interest into bacterial cells, wherein the system comprises:
i. a donor plasmid comprising a first antibiotic resistance gene, a counter selective marker and a transfer cassette, and wherein the transfer cassette comprises a left transposon arm, a gene of interest and a right transposon arm;
ii. a helper plasmid comprising a second antibiotic resistance gene;
iii. a bacmid comprising third antibiotic resistance gene and a transposon insertion site located within a reporter cassette;
(b) growing the transformed bacteria in the presence of three antibiotics to which the first, second and third antibiotic resistance genes confer resistance;
(c) selecting at least one bacterial colony comprising a recombinant bacmid;
(d) incubating bacteria from at least one bacterial colony comprising a recombinant bacmid selected in step (c) with a compound that allows counter selection against the donor plasmid;
(e) collecting recombinant bacmids comprising a gene of interest from a bacterial colony after counter selection;
(f) transforming insect cells with the recombinant bacmids comprising a gene of interest collected in step (e);
(g) incubating the transformed insect cells and harvesting rBV comprising the gene of interest from the insect cells;
(h) performing a first passage, wherein a passage comprises the steps of infecting insect cells with rBV comprising the gene of interest and incubating the infected insect cells;
(i) harvesting rBV comprising the gene of interest from one or more insect cell plaques and identifying a plaque comprising rBV comprising a stable gene of interest; and
(j) performing a second passage, wherein the second passage comprises infecting insect cells with rBV comprising a stable gene of interest, incubating the infected insect cells and harvesting a recombinant baculovirus seed stock comprising rBV comprising the stable gene of interest from the insect cells.

56. (canceled)

57. A recombinant baculovirus seed stock comprising a stable gene of interest produced by the method of claim 55.

58.-95. (canceled)

96. A method of producing rAAV comprising a gene of interest comprising the steps of:
(a) providing a first recombinant baculovirus seed stock wherein the first recombinant baculovirus seed stock comprises rBV comprising a stable first gene of interest;
(b) providing a second recombinant baculovirus seed stock, wherein the second recombinant baculovirus seed stock comprises rBV comprising a stable gene of interest encoding Rep/Cap proteins;
(c) co-infecting insect cells with the first recombinant baculovirus seed stock at a low multiplicity of infection (MOI) and with the second recombinant baculovirus seed stock at a low multiplicity of infection;
(d) adding feed to the insect cell media after co-infection;
(e) incubating the infected insect cells; and
(f) harvesting rAAV comprising the gene of interest from the insect cells.

97.-100. (canceled)

101. The system of claim 1, wherein the recombinant bacmids with reduced residual DNA are for use in rAAV production.

102. The system of claim 1, wherein the helper plasmid is an AAV helper plasmid.
